(19) United States
(12) Patent Application Publication
(10) Pub. No.: US 2003/0044447 A1
(43) Pub. Date: Mar. 6, 2003

(54) Antimicrobial contact lenses and methods for their production

(76) Inventors: Diana Zanini, Jacksonville, FL (US); Azaam Alli, Jacksonville, FL (US); James D. Ford, Orange Park, FL (US); Robert B. Steffen, Jacksonville Beach, FL (US); Douglas G. Vanderlaan, Jacksonville, FL (US); James R. Petisce, Jacksonville, FL (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/028,400

(22) Filed: Dec. 20, 2001

Related U.S. Application Data
(60) Provisional application No. 60/257,030, filed on Dec. 21, 2000.

(51) Int. Cl. A61K 9/00; G02C 7/04

(52) U.S. Cl. 424/429; 351/160 R

(57) ABSTRACT

This invention relates to antimicrobial lenses and methods for their production where the lenses contain silver and a polymerizable monomer of Formula I, II, III or IV

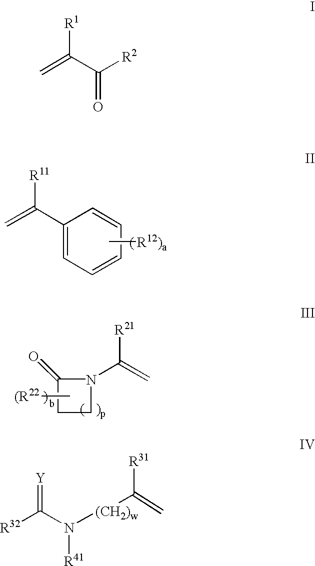

where R¹, R², R¹¹. R¹², R²¹, R²², R²², R³¹, R³², R⁴¹,Y, a, b, p, a, and w are defined herein.

### RELATED INVENTIONS

This patent application claims priority from a provisional patent application, U.S. Ser. No. 60/257,030, that was filed on Dec. 21, 2000.

### FIELD OF THE INVENTION

This invention relates to contact lenses having antimicrobial properties as well as methods of their production, use, and storage.

### BACKGROUND OF THE INVENTION

Contact lenses have been used commercially to improve vision since the 1950s. The first contact lenses were made of hard materials. Although these lenses are currently used, they are not suitable for all patients due to their poor initial comfort and their relatively low permeability to oxygen. Later developments in the field gave rise to soft contact lenses, based upon hydrogels, which are extremely popular today. Many users find soft lenses are more comfortable, and increased comfort levels allow soft contact lens users to wear their lenses for far longer hours than users of hard contact lenses.

Despite this advantage, the extended use of the lenses can encourage the buildup of bacteria or other microbes, particularly, Pseudomonas aeruginosa, on the surfaces of soft contact lenses. The build-up of bacteria or other microbes is not unique to soft contact lens wearers and may occur during the use of hard contact lenses as well.

Therefore, there is a need to produce contact lenses that inhibit the growth of bacteria or other microbes and/or the adhesion of bacterial or other microbes on the surface of contact lenses. Further there is a need to produce contact lenses which do not promote the adhesion and/or growth of bacteria or other microbes on the surface of the contact lenses. Also there is a need to produce contact lenses that inhibit adverse responses related to the growth of bacteria or other microbes.

Others have recognized the need to produce soft contact lenses that inhibit the growth of bacteria. In U.S. Pat. No. 5,213,801, the production of an antibacterial contact lens is disclosed, where an antibacterial metal ceramic material within a soft contact lens is incorporated into a contact lens. This procedure contains a number of steps and may not be suitable for producing all types of lenses in a production environment. The steps include making a silver ceramic material that is fine enough to be used in a contact lens and then forming the lens with the powdered ceramic. However, lenses containing these types of materials often lack the clarity required by contact lens users.

Although these methods and lenses are known, other contact lenses that inhibit the growth and/or adhesion of bacteria or other microbes and are of sufficient optical clarity, as well as methods of making those lenses are still needed. It is this need, which this invention seeks to meet.

### BRIEF DESCRIPTION OF THE DRAWINGS

FIG. 1 Lenses N & G Movement and Silver Concentration

FIG. 2 Lens Q Movement and Silver Concentration

### DETAILED DESCRIPTION OF THE INVENTION

This invention includes an antimicrobial lens comprising, consisting essentially of, or consisting of, silver and a polymer comprising a monomer of Formula I, II, III or IV

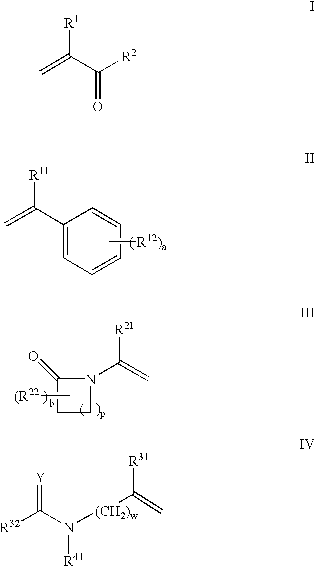

wherein

R¹ is hydrogen or C₁₋₆alkyl;

R² is —OR³, —NH—R³—S—(CH₂)_{d}—R³,or —(CH₂)_{d}—R³, wherein

d is 0-8;

R³ is substituted C₁₋₆alkyl

where the alkyl substituents are selected from one or more members of the group consisting of carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea, and substituted phenylthiourea

wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, halo C₁₋₆alkyl, halogen, hydroxyl, carboxylic acid, sulfonic acid, phosphonic acid, amine, amidine, acetamide, and nitrile;

—(CR⁴R⁵)_{q}—(CHR⁶)ₘ—SO₃H

wherein R⁴, R⁵, and R⁶ are independently selected from the group consisting of hydrogen, halogen, hydroxyl, and C₁₋₆alkyl,

q is 1-6, and

m is 0-6;

—(CH₂)ₙ—S—S—(CH₂)ₓNH—C(O)CR⁷CH₂,

wherein R⁷ is hydrogen or C₁₋₆alkyl,

n is 1-6, and

x is 1-6;

—(CR⁸R⁹)ₜ—(CHR¹⁰)ᵤ—P(O)(OH)₂

wherein R⁸, R⁹, and R¹⁰ are independently selected from the group consisting of hydrogen, halogen, hydroxyl, and C₁₋₆alkyl,

t is 1-6, and

u is 0-6;

phenyl;

benzyl;

pyridinyl;

pyrimidinyl;

pyrazinyl;

benzimidazolyl;

benzothiazolyl;

benzotriazolyl;

naphthaloyl;

quinolinyl;

indolyl;

thiadiazolyl;

triazolyl;

4-methylpiperidin-1-yl;

4-methylpiperazin-1-yl;

substituted phenyl;

substituted benzyl;

substituted pyridinyl;

substituted pyrimidinyl;

substituted pyrazinyl;

substituted benzimidazolyl;

substituted benzothiazolyl;

substituted benzotriazolyl;

substituted naphthaloyl;

substituted quinolinyl;

substituted indolyl;

substituted thiadiazolyl;

substituted triazolyl;

substituted 4-methylpiperidin-1-yl; or

substituted 4-methylpiperazin-1-yl,

wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methyl piperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl) phosphonyl, N-(amino-4-methylpiperazinyl) phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea

wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;

a is 1-5;

R¹¹ is hydrogen or C₁₋₆alkyl;

R¹² is hydroxyl, sulfonic acid, phosphonic acid, carboxylic acid, acetamide, thioC₁₋₆alkylcarbonyl, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, —OR¹³, —NH—R¹³—S—(CH₂)_{d}—R¹³, —(CH₂)_{d}—R¹³, —C(O)NH—(CH₂)_{d}—R¹³—C(O)—(CH₂)_{d}—R¹³, substituted C₁₋₆alkyidisulfide, substituted phenyldisulfide, substituted C₁₋₆alkylurea, substituted phenylurea, substituted phenylthiourea or substituted C₁₋₆alkylthiourea wherein the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile; where

d is 0-8;

R¹³ is thioC₁₋₆alkylcarbonyl;

substituted C₁₋₆alkyl

where the alkyl substituents are selected from one or more members of the group consisting of hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea and substituted phenylthiourea

wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;

—(CR¹⁴R¹⁵)_{q}—(CHR¹⁶)ₘ—SO₃H

where R¹⁴, R¹⁵, and R¹⁶ are independently selected from the group consisting of hydrogen, halogen, hydroxyl, and C₁₋₆alkyl,

q is 1-6, and

m is 0-6;

—(CH₂)ₙ—S—S—(CH₂)ₓNH—C(O)CR¹⁷CH₂,

where R¹⁷ is hydrogen or C₁₋₆alkyl,

n is 1-6, and

x is 1-6;

—(CR¹⁸ R¹⁹)ₜ—(CHR²⁰)ᵤ—P(O)(OH)₂

where R¹⁸, R¹⁹, and R²⁰ are independently selected from the group consisting of hydrogen, halogen, hydroxyl, and C₁₋₆alkyl,

t is 1-6, and

u is 0-6;

phenyl;

benzyl;

pyridinyl;

pyrimidinyl;

pyrazinyl;

benzimidazolyl;

benzothiazolyl;

benzotriazolyl;

naphthaloyl;

quinolinyl;

indolyl;

thiadiazolyl;

triazolyl;

4-methylpiperidin-1-yl;

4-methylpiperazin-1-yl;

substituted phenyl;

substituted benzyl;

substituted pyridinyl;

substituted pyrimidinyl;

substituted pyrazinyl;

substituted benzimidazolyl;

substituted benzothiazolyl;

substituted benzotriazolyl;

substituted naphthaloyl;

substituted quinolinyl;

substituted indolyl;

substituted thiadiazolyl;

substituted triazolyl;

substituted 4-methylpiperidin-1-yl; or

substituted 4-methylpiperazin-1-yl

wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methylpiperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl) phosphonyl, N-(amino-4-methylpiperazinyl) phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea

wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₁₆alkyl, halogen, hydroxyl, carboxylic acid, sulfonic acid, phosphonic acid, amine, amidine, acetamide, and nitrile;

b is 1-5;

p is 1-5;

R²¹ is hydrogen;

R²² is hydroxyl, sulfonic acid, phosphonic acid, carboxylic acid, thioC₁₋₆alkylcarbonyl, thioC₁₋₆alkylaminocarbonyl, C₁₋₆alkyldisulfide, phenyldisulfide, —C(O)NH(CH₂)₁₋₆—SO₃H, —C(O)NH(CH₂)₁₋₆—P(O)(OH)₂, —OR²³, —NH—R²³—C(O)NH—(CH₂)_{d}—R²³, —S—(CH₂)_{d}—R²³, —(CH₂)_{d}—R²³, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted, C₁₋₆alkylthiourea substituted phenylurea or substituted phenylthiourea wherein the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile, where

d is 0-8;

R²³ is thioC₁₋₆alkylcarbonyl,

C₁₋₆alkyl,

substituted C₁₋₆alkyl

where the alkyl substituents are selected from one or more members of the group consisting of C₁₋₆alkyl, halo C₁₋₆alkyl, halogen, hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆-alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea, and substituted phenylthiourea

wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;

—(CR²⁴ R²⁵)_{q}—(CHR²⁶)ₘ,SO₃H

where R²⁴, R²⁵, and R²⁶ are independently selected from the group consisting of hydrogen, halogen, hydroxyl, and C₁₋₆alkyl,

q is 1-6, and

m is 0-6

—(CH₂)ₙ—S—S—(CH₂)ₓNH—C(O)CR²⁷CH₂,

where R²⁷ is hydrogen or C₁₋₆alkyl,

n is 1-6, and

x is 1-6;

—(CR²⁸R²⁹)ₜ—(CHR³⁰)ᵤ—P(O)(OH)₂

where R²⁸, R²⁹, and R³⁰ are independently selected from the group consisting of hydrogen, halogen, hydroxyl, and C₁₋₆alkyl,

t is 1-6, and

u is 0-6;

phenyl;

benzyl;

pyridinyl;

pyrimidinyl;

pyrazinyl;

benzimidazolyl;

benzothiazolyl;

benzotriazolyl;

naphthaloyl;

quinolinyl;

indolyl;

thiadiazolyl;

triazolyl;

4-methylpiperidin-1-yl;

4-methylpiperazin-1-yl;

substituted phenyl;

substituted benzyl;

substituted pyridinyl;

substituted pyrimidinyl;

substituted pyrazinyl;

substituted benzimidazolyl;

substituted benzothiazolyl;

substituted benzotriazolyl;

substituted naphthaloyl;

substituted quinolinyl;

substituted indolyl;

substituted thiadiazolyl;

substituted triazolyl;

substituted 4-methylpiperidin-1-yl; or

substituted 4-methylpiperazin-1-yl,

wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl n yl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methylpiperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl) phosphonyl, N-(amino-4-methylpiperazinyl) phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyidisulfide, substituted phenyldisulfide, substituted C₁₋₆alkylurea, substituted C₁₋₆alkylthiourea, substituted phenylurea, and substituted phenylthiourea

wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;

w is 0-1;

Y is oxygen or sulfur;

R³¹ is hydrogen or C₁₋₆alkyl;

R³² is hydroxyl, sulfonic acid, phosphonic acid, carboxylic acid, thioC₁₋₆alkylcarbonyl, thioC₁₋₆alkylaminocarbonyl, —C(O)NH—(CH₂)_{d}—R³³, —O—R³³, —NH—R³³—S—(CH₂)_{d}—R³³, —(CH₂)_{d}—R³³, C₁₋₆alkyldisulfide, phenyldisulfide, urea, C₁₋₆alkylurea, phenylurea, thiourea, C₁₋₆alkylthiourea, phenylthiourea, C₁₋₆alkylamine, phenylamine, substituted C₁₋₆alkyldisulfide, substituted phenyldisulfide, substituted phenylurea, substituted C₁₋₆alkylamine, substituted phenylamine, substituted phenylthiourea, substituted C₁₋₆alkylurea or substituted C₁₋₆alkylthiourea wherein the substitutents are selected from the group consisting of C₁₋₆alkyl, haloC₁₋₆alkyl, halogen, hydroxyl, carboxylic acid, sulfonic acid, phosphonic acid, amine, amidine, acetamide, and nitrile

where

d is 0-8;

R³³ is thioC₁₋₆alkylcarbonyl,

C₁₋₆alkyl,

substituted C₁₋₆alkyl

where the alkyl substituents are selected from one or more members of the group consisting of C₁₋₆alkyl, halo C₁₋₆alkyl, halogen, hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea or substituted phenylthiourea

wherein the C₁₋₆alkyldisulfide, phenyldisulfide, C₁₋₁alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;

—(CR³⁴R³⁵)_{q}—(CHR³⁶)ₘ—SO₃H

where R³⁴, R³⁵, and R³⁶ are independently selected from the group consisting of hydrogen, halogen, hydroxyl, and C₁₋₆alkyl,

q is 1-6, and

m is 0-6;

—(CH₂)ₙ—S—S—(CH₂)ₓNH—C(O)CR³⁷CH₂,

where R³⁷ is hydrogen or C₁₋₆alkyl,

n is 1-6, and

x is 1-6;

—(CR³⁸R³⁹)ₜ—(CHR⁴⁰)ᵤ—P(O)(OH)₂

where R³⁸, R³⁹, and R⁴⁰ are independently selected from the group consisting of hydrogen, halogen, hydroxyl, and C₁₋₆alkyl,

t is 1-6, and

u is 0-6;

phenyl;

benzyl;

pyridinyl;

pyrimidinyl;

pyrazinyl;

benzimidazolyl;

benzothiazolyl;

benzotriazolyl;

naphthaloyl;

quinolinyl;

indolyl;

thiadiazolyl;

triazolyl;

4-methylpiperidin-1-yl;

4-methylpiperazin-1-yl;

substituted phenyl;

substituted benzyl;

substituted pyridinyl;

substituted pyrimidinyl;

substituted pyrazinyl;

substituted benzimidazolyl;

substituted benzothiazolyl;

substituted benzotriazolyl;

substituted naphthaloyl;

substituted quinolinyl;

substituted indolyl;

substituted thiadiazolyl;

substituted triazolyl;

substituted 4-methylpiperidin-1-yl; or

substituted 4-methylpiperazin-1-yl,

wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methylpiperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl) phosphonyl, N-(amino-4-methylpiperazinyl) phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea

wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;

R⁴¹ is hydrogen, C₁₋₆alkyl, phenyl, C₁₋₆alkylcarbonyl, phenylcarbonyl, substituted C₁₋₆alkyl, substituted phenyl, substituted C₁₋₆alkylcarbonyl or substituted phenylcarbonyl,

wherein

the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile.

As used herein, the term “lens” refers to opthalmic devices that reside in or on the eye. These devices can provide optical correction or may be cosmetic. The term lens includes but is not limited to soft contact lenses, hard contact lenses, intraocular lenses, overlay lenses, ocular inserts, and optical inserts. Soft contact lenses are made from silicone elastomers or hydrogels, which include but are not limited to silicone hydrogels and fluorohydrogels. These hydrogels contain hydrophobic and/or hydrophilic monomers that are covalently bound to one another in the cured lens. As used herein the term “polymers” means copolymers, homopolymers, or mixtures thereof. The monomers of Formula I, II, III or IV, or their homopolymers, are added to the monomer mix of contact lenses, prior to polymerization in an amount based on the weight percent of the initial monomer mix, including a suitable diluent if said diluent is used in the preparation of the polymer. The weight percentage of the monomers of the invention can vary with the lens formulation. The maximum percentage of monomers of Formula I, II, III or IV is the percentage that does not compromise the physical properties of the resulting contact lens, such as, but not limited to modulus, of the resulting lens. The minimum percentage of monomers of Formula I, II, III or IV is an amount that allows the incorporation of a sufficient amount of silver into a lens. Preferably, about 0.01 to about 20.0 weight percent of monomers of Formula I, II, III or IV are added, to a contact lens formulation, more preferably, about 0.01 to about 1.5 weight percent, even more preferably, about 0.01 to about 0.4 weight percent, most preferably, about 0.2 weight percent.

Monomers of Formula I, II, III or IV are added to the soft contact lens formulations described in U.S. Pat. No. 5,710,302, WO 9421698, EP 406161, JP 2000016905, U.S. Pat. No. 5,998,498, U.S. patent application Ser. No. 09/532,943 and U.S. Pat. No. 6,087,415. In addition, monomers of Formula I, II, III or IV may be added to the formulations of commercial soft contact lenses. Examples of commercially available soft contact lenses formulations include but are not limited to, the formulations of etafilcon A, genfilcon A, lenefilcon A, polymacon, acquafilcon A, balafilcon A, and lotrafilcon A. The preferable contact lens formulations are etafilcon A, balafilcon A, and silicone hydrogels, as prepared in U.S. Pat. No. 5,760,100; U.S. Pat. No. 5,776,999; U.S. Pat. No. 5,849,811; U.S. Pat. No. 5,789,461; U.S. Pat. No. 5,998,498, U.S. patent application Ser. No. 09/532,943, a continuation-in-part of U.S. patent application Ser. No. 09/532,943, filed on Aug. 30, 2000, and U.S. Pat. No. 6,087,415. These patents are hereby incorporated by reference for the hydrogel compositions contained therein. Lenses prepared from the aforementioned formulations and the monomers of Formula II, II, III or IV may be coated with a number of agents that are used to coat lenses. For example, the procedures, compositions, and methods of U.S. Pat. Nos. 3,854,982; 3,916,033; 4,920,184; and 5,002,794; 5,712,327; and 6,087,415 as well as WO 0127662, may be used and these patents are hereby incorporated by reference for those procedures, compositions, and methods. In addition to the cited coating patents, there are other methods of treating a lens once it is formed. The lenses of this invention may be treated by these methods and the following publications which illustrate these methods are hereby incorporated by reference in their entirety, U.S. Pat. No. 5,453,467; U.S. Pat. No. 5,422,402; WO 9300391; U.S. Pat. No. 4,973,493; and U.S. Pat. No. 5,350,800.

Hard contact lenses are made from polymers that include but are not limited to polymers of poly(methyl)methacrylate, silicon acrylates, fluoroacrylates, fluoroethers, polyacetylenes, and polyimides, where the preparation of representative examples may be found in JP 200010055; JP 6123860; and U.S. Pat. No. 4,330,383. Intraocular lenses of the invention can be formed using known materials. For example, the lenses may be made from a rigid material including, without limitation, polymethyl methacrylate, polystyrene, polycarbonate, or the like, and combinations thereof. Additionally, flexible materials may be used including, without limitation, hydrogels, silicone materials, acrylic materials, fluorocarbon materials and the like, or combinations thereof. Typical intraocular lenses are described in WO 0026698; WO 0022460; WO 9929750; WO 9927978; WO 0022459; and JP 2000107277. The polymerizable monomers of Formula I, II, III or IV may be added to hard contact lens formulations and intraocular lens formulations in the same manner and at the same percentage as described above for soft contact lenses. All of the references mentioned in this application are hereby incorporated by reference in their entirety.

As used herein, the term “silver” refers to silver metal that is incorporated into a lens. While not wanting to be bound as to the oxidation state of the silver (Ag⁰, Ag¹⁺, or Ag²⁺), that is incorporated into the lens, silver may be added to the lens by washing the cured and hydrated lens in a silver solution such as silver nitrate in deionized water (“DI”). Other sources of silver include but are not limited to silver acetate, silver citrate, silver iodide, silver lactate, silver picrate, and silver sulfate. The concentration of silver in these solutions can vary from the concentration required to add a known quantity of silver to a lens to a saturated silver solution. In order to calculate the concentration of the silver solution needed, the following calculation is used: the concentration of silver solution is equal to the desired amount of silver per lens, multiplied by the dry weight of the lens divided by the total volume of treating solution.

silver solution concentration (μg/mL)=[desired silver in lens (μg/g)×average dry lens weight (g)]/total volume of treating solution (mL)

For example, if one requires a lens containing 40 μg/g of silver, the dry weight of the lens is 0.02 g, and the vessel used to treat said lens has a volume of 3 mL, the required silver concentration would be 0.27 μg/mL.

Silver solutions containing anywhere from about 0.10 μg/mL to 0.3 grams/mL have been used to prepare the lenses of the invention. Aside from deionized water, other liquid mediums can be used such as water, aqueous buffered solutions and organic solutions such as polyethers or alcohols. Typically, the lens is washed in the silver solution for about 60 minutes, though the time may vary from about 1 minute to about 2 hours and at temperatures ranging from about 5° C. to about 130° C. After the silver treatment the lenses are washed with several portions of water to obtain a lens where silver is incorporated into the polymer. The amount of silver that is incorporated into the lenses ranges from about 20 ppm to about 100,000 ppm, where any lens containing at least about 20 ppm has antimicrobial properties. The preferred amount of silver that is incorporated into the lens is about 20 ppm to about 4,000 ppm, more preferably, 20 ppm to about 1,500 ppm, even more preferably about 30 ppm to about 600 ppm, and most preferably about 30 ppm to about 75 ppm.

The term “antimicrobial” refers to a lens that exhibit one or more of the following properties—the inhibition of the adhesion of bacteria or other microbes to the lenses, the inhibition of the growth of bacteria or other microbes on the lenses, and the killing of bacteria or other microbes on the surface of the lenses or in a radius extending from the lenses (hereinafter adhesion of bacteria or other microbes to the lenses, the growth of bacteria or other microbes to the lenses and the presence of bacterial or other microbes on the surface of lenses is collectively referred to as “microbial production”). The lenses of the invention inhibit the microbial production by at least 25%. Preferably, the lenses of the invention exhibit at least a 1-log reduction (≧90% inhibition) of viable bacteria or other microbes, more preferably a 2-log reduction (≧99% inhibition) of viable bacteria or other microbes. Such bacteria or other microbes include but are not limited to those organisms found in the eye, particularly Pseudomonas aeruginosa, Acanthamoeba species, Staphyloccus. aureus, E. coli, Staphyloccus epidermidis, and Serratia marcesens. Preferably, said antimicrobial lens is a clear lens, that has clarity comparable to currently available commercial lenses such as but not limited to, etafilcon A, genfilcon A, lenefilcon A, polymacon, acquafilcon A, balafilcon A, and lotrafilcon A.

The term “phosphonyl” refers to a radical having the following structure

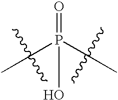

With respect to the monomers of Formula I, there are some monomers that are preferred. The preferred monomers of Formula I include monomers where R¹ is hydrogen or C₁₋₃alkyl;

R²is NH—R³;

d is 0

R³ is substituted phenyl, —(CR⁴, R⁵)_{q}—(CHR⁶)ₘ,SO₃H, —(CR⁸R⁹)ₜ—(CHR¹⁰)ᵤ—P(O)(OH)₂, or —(CH₂)ₙ—S—S—(CH₂)ₓNH—C(O)CR⁷CH₂;

R⁴ is hydrogen or C₁₋₃alkyl;

R⁵ is hydrogen or C₁₋₃alkyl;

R⁶ is hydrogen or C₁₋₃alkyl;

q is 1-3;

m is 1-3;

R⁷ is hydrogen or C₁₋₃alkyl;

R⁸ is hydrogen or C₁₋₃alkyl;

R⁹ is hydrogen or C₁₋₃alkyl;

R¹⁰ is hydrogen or C₁₋₃alkyl;

t is 1-3;

u is 1-3;

n is 2-4; and

x is 2-4.

The more preferred monomers of Formula I include monomer where

R¹ is hydrogen or methyl;

R² is NH—R³;

R³ is —(CR⁴R⁵)_{q}—(CHR⁶)ₘSO₃H, —(CR⁸R⁹)ₜ—(CHR¹⁰)ᵤ—P(O)(OH)₂ or —(CH₂)ₙ—S—S—(CH₂)ₓNH—C(O)CHR⁷CH₂;

R⁴ is hydrogen or methyl;

R⁵ is hydrogen or methyl;

q is 1-2;

m is 1-2;

R⁶ is hydrogen or methyl;

R⁷ is hydrogen;

R⁸ is hydrogen or methyl;

R⁹ is hydrogen or methyl;

R¹⁰ is hydrogen or methyl;

t is 1;

u is 1-2;

n is 2-3; and

x is 2-3.

The most preferred monomers of Formula I include the following monomers

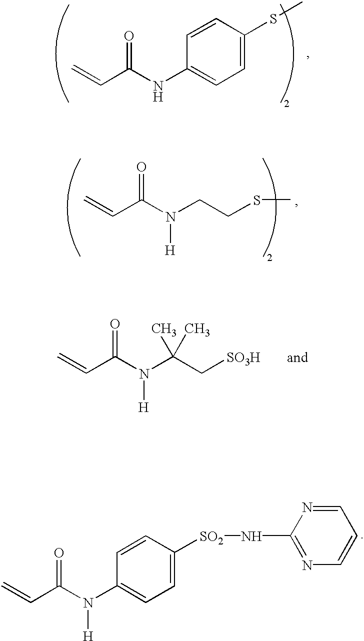

With respect to the monomers of Formula II, there are some monomers that are preferred. The preferred monomers of Formula II include monomers where

a is 1-2;

R¹¹ is hydrogen or C₁₋₃alkyl;

R¹² is sulfonic acid, carboxylic acid, phosphonic acid, C₁₋₆alkyldisulfide, C₁₋₆alkylsulfide, phenyldisulfide, substiuted phenyldisulfide or NH—R¹³;

R¹³ is thioC₁₋₆alkylcarbonyl.

The most preferred monomers of Formula II include the following monomers

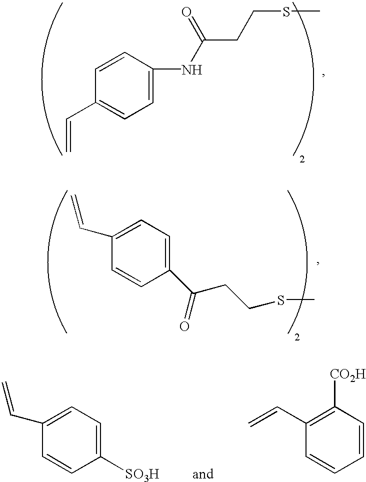

With respect to the monomers of Formula II, there are some monomers that are preferred. The preferred monomers of Formula III include monomers where

p is 1-3;

b is 1-2;

R²¹ is hydrogen;

R²² is sulfonic acid, phosphonic acid, carboxylic acid, thioC₁₋₆alkylcarbonyl, thioC₁₋₆alkylaminocarbonyl, C₁₋₆alkyldisulfide, C₁₋₆alkylsulfide, phenyldisulfide, substiuted phenyldisulfide, H₃OS—(CH₂)₁₋₆NHC(O) or (HO)₂(O)P—(CH₂)₁₋₆NHC(O)—.

The most preferred monomers of Formula III include the following monomers

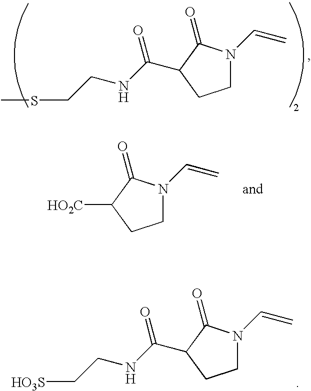

With respect to the monomers of Formula IV, there are some monomers that are preferred. The preferred monomers of Formula IV include monomers where

w is 0-1;

R³¹ is hydrogen;

R³² is amine, C₁₋₃alkylamine, phenylamine, substituted phenylamine, thioC₁₋₃alkylcarbonyl;

R⁴¹ is hydrogen

The most preferred monomers of Formula IV include the following monomers

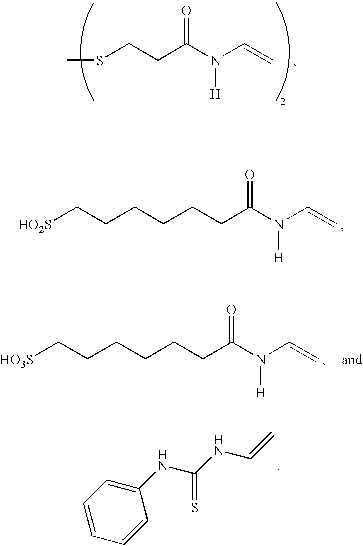

For monomers of all formula, the preferred points of attachment are as follows pyridinyl is 4, for pyrimidinyl is 2, for benzimidazolyl is 2, for benzothiazole is 2, for benzotriazolyl is 5, for quinolinyl is 2, for indolyl is 4 or 5, for thiadiazole is 3, or 5, and for triazolyl is 3 or 5, where positions containing hydrogen may be substituted with the named substituents.

Further, the invention includes an antimicrobial lens comprising, consisting essentially of, or consisting of, silver and a polymer comprising a binding monomer where said cured lens can reversibly bind silver. The terms antimicrobial, lens, and silver all have their aforementioned meanings and preferred ranges. The term “binding monomer” means any polymerizable monomer that can reversibly bind silver. The term “cured lens” refers to contact lens monomer formulations polymerized with binding monomers. The potential ability of a cured lens to reversibly bind silver can be estimated by examining the stability constant of the selected binding monomers. These estimates can be determined by known methods. (See R. I. Tilley, Aust J. Chem. 1990, 43,1573). The log of the stability constants, βₙ, of the cured lense of the invention are about 0.6 to about 15.0; that is, logβₙ=[AgLₙ⁺]/([Ag⁺][L]ⁿ) where βₙ=stability constant for a cured lens (L) that binds n molecules of silver (Ag⁺), preferably, about 2 to about 7.3, and more preferably about 3.6 to 6.9.

The advantages of the antimicrobial lenses of the invention are many. For example, other antimicrobial lenses that incorporate silver usually contain silver coordinated to some inorganic particulate matter (see U.S. Pat. No. 5,213,801, discussing the use of silver ceramics). Often that particulate matter is visible to the naked or magnified eye, and it can affect the visual acuity of the user. However, the lenses of the invention do not have this problem. The monomers of Formula I, II, III or IV and other binding monomers are generally soluble with all of the other components of the antimicrobial lenses. Therefore when the lenses are produced they do not have substantial particulate matter due to their antimicrobial components. The antimicrobial lenses of the invention have comparable clarity to commercial lenses such as etafilicon A, genfilcon A, lenefilcon A, polymacon, acquafilcon A, balafilcon A, and lotrafilcon A.

Further, the invention includes a method of producing an antimicrobial lens comprising, silver and a polymer comprising a monomer of Formula I, II, III or IV

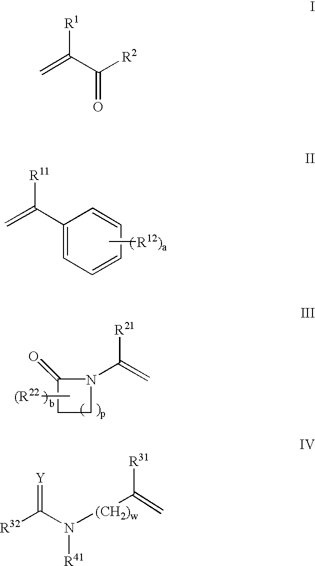

wherein R¹-R⁴¹, Y, a, q, m, n, p, d, b, t, u, w, and x are as described above wherein

the method comprises, consists essentially of, or consists of the steps of

(a) preparing a lens comprising a monomer of Formula I, II, III or IV and

(b) treating said lens with a silver solution.

The terms lens, antimicrobial, lens, silver, R¹-R⁴¹, Y, a, q, m, n, p, d, b, t, u, w, and x, all have their aforementioned meanings and preferred ranges. The term, “silver solution” refers to any liquid medium containing silver. The liquid medium includes but is not limited to water, deionized water, aqueous buffered solutions, alcohols, polyols, and glycols, where the preferred medium is deionized water. The silver of the solution is typically a silver salt such as silver nitrate, silver acetate, silver citrate, silver iodide, silver lactate, silver picrate, and silver sulfate. The concentration of silver in these solutions can vary from the concentration required to add a known quantity of silver to a lens to a saturated silver solution. In order to calculate the concentration of the silver solution needed, the following calculation is used: the concentration of silver solution is equal to the desired amount of silver per lens, multiplied by the dry weight of the lens divided by the total volume of treating solution.

silver solution concentration (μg/mL)=[desired silver in lens (μg/g)×average dry lens weight (g)]/total volume of treating solution (mL)

For example, if one requires a lens containing 40 μg/g of silver, the dry weight of the lens is 0.02 g, and the vessel used to treat said lens has a volume of 3 mL, the required silver concentration would be 0.27 μg/mL.

Silver solutions containing anywhere from about 0.10 μg/mL to 0.3 grams/mL have been used to prepare the lenses of the invention. Aside from deionized water, other liquid mediums can be used such as water, aqueous buffered solutions and organic solutions such as polyethers, or alcohols. Typically, the lens is washed in the silver solution for about 60 minutes, though the time may vary from about 1 minute to about 2 hours and at temperatures ranging from about 5° C. to about 130° C. After the silver treatment the lenses are washed with several portions of water to obtain a lens where silver is incorporated into the polymer.

Still further, the invention includes a lens case comprising, consisting essentially of, or consisting of silver and a polymer of a monomer of Formula I, II, III or IV

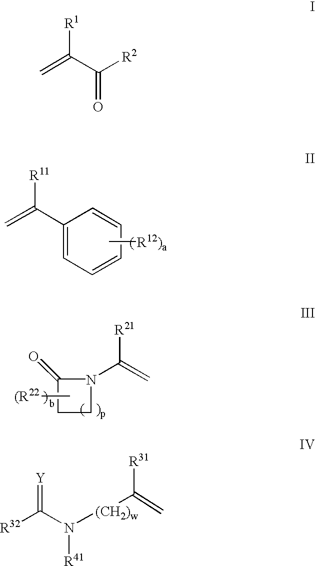

wherein R¹-R⁴¹, Y, a, q, m, n, p, d, b, t, u, w, and x are as described above

The terms lens, silver, R¹-R⁴¹, Y, a, q, m, n, p, d, b, t, u, w, and x, all have their aforementioned meanings and preferred ranges. The term lens case refers to a container that is adapted to define a space in which to hold a lens when that lens is not in use. This term includes packaging for lenses, where packaging includes any unit in which a lens is stored after curing. Examples of this packaging include but are not limited to single use blister packs, multiple use storage cases and the like.

One such container is illustrated in FIG. 3 of U.S. Pat. No. 5,515,117 which is hereby incorporated by reference in its entirety. The polymers of Formula I, II, III or IV can be incorporated in the lens container 22, the cover 24, or the lens basket 26, where they are preferably incorporated into the lens container or the lens basket.

Aside from the polymers of Formula I, II, III or IV the container components may be made of a transparent, thermo-plastic polymeric material, such as polymethylmethacrylate, polyolefins, such as poly-ethylene, polypropylene, their copolymers and the like; polyesters, polyurethanes; acrylic polymers, such as polyacrylates and polymethacrylates; polycarbonates and the like and is made, or any combination thereof, e.g., molded, using conventional techniques as a single unit.

Silver may be incorporated into the lens container in the same manner that it is incorporated into the antimicrobial lenses of the invention. More specifically, the polymers of Formula I, II, III or IV are combined with the formulation of the other components, molded, cured, and subsequently treated with a silver solution. Preferably, polymers of Formula I, II, III or IV are present in any or all of the lens case components at about 0.01 to about 10.0 weight percent (based on the initial monomer mix), more preferably about 0.01 to about 1.5 percent. Storing lenses in such an environment inhibits the growth of bacteria on said lenses and adverse effects that are caused by the proliferation of bacterial. Another example of such a lens case is the lens case can be found in U.S. Pat. No. 6,029,808 which is hereby incorporated by reference for the blister pack housing for a contact lens disclosed therein.

Yet still further, the invention includes a method of reducing the adverse effects associated with microbial production in the eye of a mammal, comprising, consisting essentially of, or consisting of providing an antimicrobial lens wherein said lens comprises silver and a polymer of a monomer of Formula I, II, III or IV

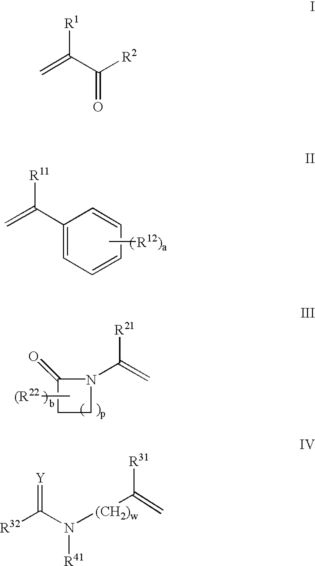

wherein R¹-R⁴, Y, a, q, m, n, p, d, b, t, u, w, and x are as described above

The terms lens, antimicrobial, lens, silver, R¹-R⁴¹, Y, a, q, m, n, p, d, b, t, u, w and x, all have their aforementioned meanings and preferred ranges. The phrase “adverse effects associated with microbial production” includes but is not limited to, ocular inflammation, contact lens related peripheral ulcers, contact lens associated red eye, infiltrative keratitis, and microbial keratitis.

Providing a lens that fits a wide range of patients has been a quest of eye care practitioners and lens manufactures for a number of years. In order to produce such a lens, many variables, such as lens material, design, surface treatments, and additional components such as ophthalmic drugs, tints, dyes and pigments can come into play. For example it has been shown that if one adds too much of an additional component, such as an antimicrobial agent, a lens that will become adhered to the eye is produced. However, if one is attempting to produce an antimicrobial lens, a balance should be struck between producing a lens that contains enough antimicrobial agent to produce the desired effect without producing a lens that adheres to the eye.

One way to assess if a lens fit is acceptable (i.e. the lens is not adhered) is to assess the tightness of the fit of a lens. (Young, G. et al., Influence of Soft Contact Lens Design on Clinical Performance, Optometry and Vision Science, Vol 70, No., 5 pp. 394-403) Tightness of a lens may be assessed using an in vivo push up test. In that test, a lens is placed on a patient's eye. Subsequently, an eye care practitioner presses his or her finger digitally upward against the lower lid of the patient's eye and observes whether the lens moves on the patient's eye (Id.). Lenses that do not move under these circumstances are not considered to be a good fit for the patient's eye, for lenses that are too tight will not move when the patient blinks and may become uncomfortable. Therefore one of the objects of this invention is to produce an antimicrobial lens that does not adhere to the patient's eye.

To meet this objective, the invention includes an antimicrobial lens comprising, consisting essentially of, or consisting of silver, wherein said lens has sufficient movement on the eye of a patient. The terms lens, antimicrobial, lens, silver, R¹-R⁴¹, Y, a, q, m, n, p, d, b, t, u, w, and x, all have their aforementioned meanings and preferred ranges. The phrase “movement on the eye of a patient” refers to whether a lens, when placed on the eye of a patient moves under the push-up test described above. This test is described in further detail in Contact Lens Practice, Chapman & Hall, 1994, edited by M. Ruben and M. Guillon, pgs. 589-99. Under this test lenses are given an −2 rating if they do not move on the eye of a patient in the digital push-up test. Therefore lenses that score greater than a “−2” on the digital push-up test are lenses that move on a patient's eye. In a statistically significant patient population, lenses that may be suitable for one patient may not be suitable for another. Therefore, lenses having sufficient movement are lenses that move on at least about 50 to about 100% of a given patient population. Preferably, said lenses move on about 75 to about 100%, of patients, more preferably, about 80 to about 100%, most preferably about 90 to about 100%.

The lenses of the invention are one method of making lenses that contain silver and have sufficient movement on the eye of a patient; however, they are not only lenses containing silver that may have sufficient movement. Other methods of incorporating into contact lenses may be used, provided that those methods produce lenses having sufficient movement on the eye of a patient.

In order to illustrate the invention the following examples are included. These examples do not limit the invention. They are meant only to suggest a method of practicing the invention. Those knowledgeable in contact lenses as well as other specialties may find other methods of practicing the invention. However, those methods are deemed to be within the scope of this invention.

### EXAMPLES

The following abbreviations were used in the examples

APDS=acrylamidophenylsulfide

AMPSA=2-acrylamido-2-methyl-1-propanesulfonic acid;

CYST=N,N′-(bisacryloyl)cystamine;

PVP=polyinylpyrrolidinone;

MM=methacrylic acid;

PAA=poly(acrylic acid)

ATU=allylthiourea;

VIM=vinylimidazole;

MABP=methacrylamido bipyrimidine;

MAHB=4-methacryloxy-2-hydroxybenzophenone;

PSPM=N-[p-(N-pyrimidin-2-sulfamoyl)phenyl]methacrylamide

Cell/prot=(Acrylamidomethyl)cellulose acetate propionate

3M3P=3-methyl-3-propanol

D3O=3,7-dimethyl-3-octanol

TAA=t-amyl alcohol

BAGE=glycerin esterified with boric acid

DI=deionized water;

PBS=phosphate-buffered saline, pH 7.4±0.2;

TPBS=Phosphate-buffered saline with 0.05% Tween™ 80, pH 7.4±0.2;

TSA=sterile tryptic soy agar;

TSB=sterile tryptic soy broth;

60% IPA=isopropyl alcohol, 60% v/v DI;

70% IPA=isopropyl alcohol, 70% v/v DI;

10% IPA=isopropyl alcohol, 10% v/v DI;

MVD=modified vortex device;

TBACB=tetrabutyl ammonium-m-chlorobenzoate

TMI=dimethyl meta-isopropenyl benzyl isocyanate

MMA=methyl methacrylate

HEMA=hydroxyethyl methacrylate

Bloc-HEMA=2-(trimethylsiloxy) ethyl methacrylate

TRIS=tris(trimethylsiloxy)-3-methacryloxypropylsilane

mPDMS=mono-methacryloxypropyl terminated polydimethylsiloxane MW=800-1000

DMA=N,N-dimethylacrylamide

Blue HEMA=the reaction product of reactive blue number 4 and HEMA as described in Example 4 of U.S. Pat. No. 5,944,853

DAROCUR 1173=2-hydroxy-2-methyl-1-phenyl-propan-1-one

EGDMA=ethyleneglycol dimethacrylate

TMPTMA=trimethyloyl propane trimethacrylate

TEGDMA=tetraethyleneglycol dimethacrylate

Norbloc=2-(2′-hydroxy-5-methacrylyloxyethylphenyl)-2H-benzotriazole

CGI 1850=1:1 (w/w) blend of 1-hydroxycyclohexyl phenyl ketone and bis (2,6-dimethyoxybenzoyl)-2,4-4-trimethylpentyl phosphine oxide

THF=tetrahydrofuran

HAM=hydroxyalkylmethacrylate, as described in U.S. Pat. No. 5,98,498

w/w=weight/total weight

w/v=weight/total volume

v/v=volume/total volume

pHEMA=poly(hydroxyethyl) methacrylate coating as described in Example 14 of U.S. Ser. No. 09/921,192, “Methods for Coating Articles by Mold Transfer”

The contact lenses of the invention were evaluated using the following biological assay, where Test B is the preferred method for determining inhibition of microbial production under the present invention.

Test A Inhibition of Bacterial Growth/Adhesion

A culture of Pseudomonas aeruginosa, ATCC# 15442 (ATCC, Rockville, Md.) was grown overnight in a nutrient medium. The bacterial inoculum was prepared to result in a final concentration of approximately 1×10⁸ colony forming units/mL. Three contact lenses were rinsed with phosphate buffered saline (PBS) pH 7.4±0.2. Each rinsed contact lens was combined with two (2) mL of bacterial inoculum into a sterile glass vial, which was rotated in a shaker-incubator (100 rpm) for two (2) hrs. at 37±2° C. Each lens was rinsed with PBS to remove loosely bound cells, placed into 10 mL of PBS containing 0.05% w/v Tween™ 80 and vortexed at 2000 rpm for three minutes. The resulting supernatant was enumerated for viable bacteria, and the results, reported of the detected viable bacteria attached to three lenses were averaged.

Test B Inhibition of Bacterial Growth/Adhesion

A culture of Pseudomonas aeruginosa, ATCC#15442 (ATCC, Rockville, Md.) was grown overnight in a nutrient medium. The bacterial inoculum was prepared to result in a final concentration of approximately 1×10⁶ colony forming units/mL. Three contact lenses were rinsed with phosphate buffered saline (PBS) pH 7.4±0.2. Each rinsed contact lens was combined with two (2) mL of bacterial inoculum into a sterile glass vial, which was rotated in a shaker-incubator (100 rpm) for 24 hr. at 35±2° C. Each lens was rinsed with PBS to remove loosely bound cells, placed into 10 mL of PBS containing 0.05% w/v Tween™ 80 and vortexed at 2000 rpm for three minutes. The resulting supernatant was enumerated for viable bacteria, and the results, reported of the detected viable bacteria attached to three lenses were averaged.

The formulations that were used to prepare the lenses of the invention were prepared as follows.

Macromer 2 Preparation

To a dry container housed in a dry box under nitrogen at ambient temperature was added 30.0 g (0.277 mol) of bis(dimethylamino)methylsilane, a solution of 13.75 mL of a 1 M solution of TBACB (386.0 g TBACB in 1000 mL dry THF), 61.39 g (0.578 mol) of p-xylene, 154.28 g (1.541 mol) methyl methacrylate (1.4 equivalents relative to initiator), 1892.13 (9.352 mol) 2-(trimethylsiloxy)ethyl methacrylate (8.5 equivalents relative to initiator) and 4399.78 g (61.01 mol) of THF. To a dry, three-necked, round-bottomed flask equipped with a thermocouple and condenser, all connected to a nitrogen source, was charged the above mixture prepared in the dry box.

The reaction mixture was cooled to 15° C. while stirring and purging with nitrogen. After the solution reached 15° C., 191.75 g (1.100 mol) of 1-trimethylsiloxy-1-methoxy-2-methylpropene (1 equivalent) was injected into the reaction vessel. The reaction was allowed to exotherm to approximately 62° C. and then 30 mL of a 0.40 M solution of 154.4 g TBACB in 11 mL of dry THF was metered in throughout the remainder of the reaction. After the temperature of reaction reached 30° C. and the metering began, a solution of 467.56 g (2.311 mol) 2-(trimethylsiloxy)ethyl methacrylate (2.1 equivalents relative to the initiator), 3636.6. g (3.463 mol) n-butyl monomethacryloxypropyl-polydimethylsiloxane (3.2 equivalents relative to the initiator), 3673.84 g (8.689 mol), TRIS (7.9 equivalents relative to the initiator) and 20.0 g bis(dimethylamino)methylsilane was added.

The mixture was allowed to exotherm to approximately 38-42° C. and then allowed to cool to 30° C. At that time, a solution of 10.0 g (0.076 mol) bis(dimethylamino)methylsilane, 154.26 g (1.541 mol) methyl methacrylate (1.4 equivalents relative to the initiator) and 1892.13 g (9.352 mol) 2-trimethylsiloxy)ethyl methacrylate (8.5 equivalents relative to the initiator) was added and the mixture again allowed to exotherm to approximately 40° C. The reaction temperature dropped to approximately 30° C. and 2 gallons of THF were added to decrease the viscosity. A solution of 439.69 g water, 740.6 g methanol and 8.8 g (0.068 mol) dichloroacetic acid was added and the mixture refluxed for 4.5 hours to de-block the protecting groups on the HEMA. Volatiles were then removed and toluene added to aid in removal of the water until a vapor temperature of 110° C. was reached.

The reaction flask was maintained at approximately 110° C. and a solution of 443 g (2.201 mol) TMI and 5.7 g (0.010 mol) dibutyltin dilaurate were added. The mixture was reacted until the isocyanate peak was gone by IR. The toluene was evaporated under reduced pressure to yield an off-white, anhydrous, waxy reactive monomer. The macromer was placed into acetone at a weight basis of approximately 2:1 acetone to macromer. After 24 hrs, water was added to precipitate out the macromer and the macromer was filtered and dried using a vacuum oven between 45 and 60° C. for 20-30 hrs.

Macromer 1 Preparation

The procedure for Macromer 2 used except that 19.1 mole parts HEMA, 5.0 mole parts MAA, 2.8 mole parts MMA; 7.9 mole parts TRIS, 3.3, mole parts mPDMS, and 2.0 mole parts TMI were used.

Macromer 3 Preparation

The procedure for Macromer 2 was used except that 19.1 mole parts HEMA, 7.9 mole parts TRIS, 3.3 mole parts mPDMS, and 2.0 mole parts TMI were used.

Marcromer 4 Preparation

The procedure for Macromer 2 was used except that dibutyltin dilaurate was replaced with triethylamine.

Base Monomer Formations and Lens Preparation

Formulations A—R, listed in Table 1, are representative base monomer mixes (all amounts are calculated as weight percent of the total weight of the combination). The polymerizable monomers of the invention are added to these mixtures as indicated in Table 2 and contact lenses are prepared according to the following method.

Contact lenses are prepared by adding the indicated amount of the polymerizable monomer to about 10 g of the base monomer mix in the presence of 1-5% wt acetic acid (when Marcromer 4 is used, no acetic acid is added) and a diluent suitable for compatiblizing the components, as indicated in Table 1. This mixture issonicated at 25-37° C. until all components are dissolved (30-120 minutes) and was subsequently loaded into an eight cavity lens mold of the type described in U.S. Pat. No. 4,640,489 and cured for 1200 sec at temperatures of, but not limited to, 25 to 90° C., preferably between 45 to 75° C. Polymerization occurred under a nitrogen purge and was either photoinitiated with 5 mW cm⁻² of UV light generated with an Andover Corp. 420PS10-25 AM39565-02 light filter, or photoinitiated with visible light generated with a Philips TL 20W/03T fluorescent bulb. The time of curing varied from 7 minutes to 60 minutes. After curing, the molds are opened, and the lenses are either released in a 1:1 blend of water and ethanol, then leached in ethanol to remove any residual monomers and diluent, or released in a 60% PA/water, then leached in IPA/DI to remove any residual monomers and diluent. Finally the lenses were equilibrated in either physiological borate-buffered saline or de-ionized water.

TABLE 1

| Formulation | A | B | C | D | E | F | G | H | I |
|---|---|---|---|---|---|---|---|---|---|
| Macromer | 1 | 2 | 3 | 3 | 2 | 2 | 2 | 2 | 2 |
| [Macromer] | 30.00 | 25.00 | 60.00 | 20.00 | 17.98 | 17.98 | 18.00 | 19.98 | 17.98 |
| TRIS | 0.00 | 18.00 | 0.00 | 40.00 | 21.00 | 21.00 | 14.00 | 8.00 | 20.00 |
| DMA | 27.00 | 28.00 | 36.00 | 36.00 | 25.50 | 25.50 | 26.00 | 26.00 | 22.00 |
| mPDMS | 39.00 | 18.00 | 0.00 | 0.00 | 21.00 | 21.00 | 28.00 | 28.50 | 25.50 |
| Norbloc. | 2.00 | 2.00 | 3.00 | 3.00 | 2.00 | 2.00 | 2.00 | 2.00 | 2.00 |
| CGI 1850 | 2.00 | 1.00 | 1.00 | 1.00 | 1.00 | 1.00 | 1.00 | 1.00 | 1.00 |
| TEGDMA | 0.00 | 0.00 | 0.00 | 0.00 | 1.50 | 1.50 | 1.00 | 1.50 | 1.50 |
| HEMA | 0.00 | 0.00 | 0.00 | 0.00 | 5.00 | 5.00 | 5.00 | 5.00 | 5.00 |
| Blue HEMA | 0.00 | 0.00 | 0.00 | 0.00 | 0.02 | 0.02 | 0.02 | 0.02 | 0.02 |
| PVP | 0.00 | 8.00 | 0.00 | 0.00 | 5.00 | 5.00 | 5.00 | 8.00 | 5.00 |
| Darocur | | | | | | | | | |
| 1173 | | | | | | | | | |
| EGDMA | | | | | | | | | |
| TMPTMA | | | | | | | | | |
| MAA | | | | | | | | | |
| Diluent % | 41 | 20 | 20 | None | 20 | 50.00 | 20.00 | 37.50 | 20.00 |
| Diluent | 3M3P | 3M3P | 3M3P | NA | D30 | TAA | D30 | 3M3P | TAA |

| Formulation | J | K | M | N | O | P | Q | R |
|---|---|---|---|---|---|---|---|---|
| Macromer | 2 | 2 | 2 | 2 | 2 | 2 | 2 | 2 |
| [Macromer] | 17.98 | 19.98 | 40.00 | 18.00 | 18.00 | 18.00 | | |
| TRIS | 25.00 | 20.00 | 20.00 | 14.00 | 14.00 | 14.00 | | |
| DMA | 9.00 | 23.00 | 35.00 | 26.00 | 26.00 | 26.00 | | |
| mPDMS | 30.00 | 28.50 | | 28.00 | 28.00 | 28.00 | | |
| Norbloc. | 2.00 | 2.00 | 3.00 | 2.00 | 2.00 | 2.00 | | |
| CGI 1850 | 1.00 | 1.00 | 2.00 | 1.00 | 1.00 | 1.00 | | |
| TEGDMA | 0.50 | 1.50 | | | 0.25 | 0.50 | | |
| HEMA | 7.00 | 5.00 | | 5.00 | 5.00 | 5.00 | 96.8 | 98.6 |
| Blue HEMA | 0.02 | 0.02 | | | | | | |
| PVP | 7.50 | 9.00 | | 5.00 | | | | |
| Darocur | | | | | | | 0.3 | 0.30 |
| 1173 | | | | | | | | |
| EGDMA | | | | | | | 0.8 | 0.8 |
| TMPTMA | | | | | | | 0.1 | 0.1 |
| MAA | | | | | | | 2.0 | |
| Diluent % | 40.00 | 50.00 | 20.00 | 20.00 | 20.00 | 20.00 | 52.00 | 52.00 |
| Diluent | 3M3P | 3M3P | D30 | D30 | D30 | D30 | BAGE | BAGE |

### Example 1

### Preparation of Antimicrobial Contact Lenses Using PSPM

Contact lenses prepared from PSPM (2365 ppm or 0.24 weight percent) and base monomer mix (Table 1,10.0 g). were treated with a 10% w/v solution of AgNO₃ in deionized water for about 60 minutes (30 lenses in 60 mL of 10 wt. % AgNO₃ in deionized water). The treated lenses were removed from the silver solution and placed into distilled water (300 mL). The lenses were either rolled or stirred in distilled water for at least about 20 minutes. This water washing procedure was repeated three (3) more times. The resulting lenses were stored in saline solution and tested to determine their antimicrobial potential. The results of the bacterial adhesion assay are presented in Table 2. In addition, the lenses were analyzed by inductively coupled argon plasma atomic emission spectroscopy (ICP-AES) of a hydrogenfluoride (HF) digest of a dry lens or using instrumental neutron activation analysis, to determine the amount of silver that was incorporated in the lenses. This data is presented in Table 2.

### Example 2

### Preparation of Antimicrobial Contact Lenses Using Polymerizable Monomers other than PSPM

The procedure of Example 1 was repeated replacing the amount of polymerizable ligand and the base monomer mix as indicated by Table 2. Table 2 lists, the Base Monomer Mix (from Table 1); the polymerizable monomer of Formula I; the concentration of the polymerizable monomer of Formula I in ppm; the amount of silver incorporated into the lens; the % inhibition from the bacterial assay, using lenses made from formulation Q without any added polymerizable monomer as the control; the % inhibition from the bacterial assay using lenses made from formulation G as the control. (Lenses were tested by inductively coupled plasma atomic emission spectroscopy). The antimicrobial activity of formulation Q lenses and formulation G lenses are statistically the same (95% confidence (p=0.05)). In the Base Monomer Mix column, the “Ag” suffix indicates that the lenses were treated with 10% AgNO₃ as described in Example 1.

TABLE 2

| Base | Monomer of | [Formula I] | [Ag], | % Inhibition | % Inhibition | Trial |
|---|---|---|---|---|---|---|
| Monomer Mix | Formula I | ppm | ppm | Q | G | no. |
| Q-Ag | PSPM | 2365 | N/A | 96.68 | N/A | |
| G | PSPM | 2365 | ** | 75.83 | 52.99 | 1 |
| G | PSPM | 2365 | ** | 60.64 | 42.55 | 2 |
| G-Ag | PSPM | 2365 | 265 | 99.64 | 99.30 | 1 |
| G-Ag | PSPM | 2365 | N/A | 97.74 | 96.71 | 2 |
| G | ATU | 438 | ** | 20.00 | N/A | |
| G-Ag | ATU | 438 | N/A | 45.00 | N/A | |
| G | ATU | 2800 | ** | 0.00 | N/A | |
| G-Ag | ATU | 2800 | 2700 | 0.00 | N/A | |
| G | VIM | 1124 | ** | 40.00 | N/A | |
| G-Ag | VIM | 1124 | N/A | 32.65 | N/A | |
| R-Ag | MAA | 9,000 | 15 | 0.00 | 0.00 | |
| R-Ag | MAA | 18,000 | 550 | 36.85 | 38.38 | |
| R-Ag | MAA | 36,000 | 1100 | 94.89 | 95.01 | |
| G | MAA | 18,000 | ** | 60.53 | 46.43 | |
| G | MAA | 27,000 | ** | 36.84 | 14.29 | |
| G | MAA | 36,000 | ** | 1.62 | 0.00 | |
| G-Ag | MAA | 5,000 | N/A | 46.17 | 47.48 | |
| G-Ag | MAA | 18,000 | N/A | 91.34 | N/A | |
| G-Ag | MAA | 27,000 | N/A | 93.00 | N/A | |
| G-Ag | MAA | 36,000 | 1800 | 91.50 | N/A | |
| G | MAHB | 3610 | ** | 26.32 | N/A | |
| G | MAHB | 16,000 | ** | 27.32 | N/A | |
| G-Ag | MAHB | 3610 | 1.9 | 58.70 | N/A | |
| G-Ag | MAHB | 16,000 | N/A | 13.48 | 35.15 | |
| G-Ag | MAHB* | 16,000 | 310 | 8.36 | 0.00 | |
| G | MABP | 2512 | ** | 45.22 | 21.82 | |
| G | MABP | 89,000 | ** | 0.00 | 14.34 | |
| G-Ag | MABP | 2512 | N/A | 53.08 | 33.03 | |
| G-Ag | MABP | 89,000 | 1.9 | 12.13 | 34.14 | |
| G-Ag | MABP* | 89,000 | 61 | 70.03 | 54.38 | |
| G | CELL/prot | 10,000 | ** | 38.30 | 58.99 | |
| G-Ag | CELL/prot | 10,000 | 3.4 | 40.73 | 60.61 | |
| G | AMPSA | 500 | ** | 43.17 | 0.00 | 1 |
| G | AMPSA | 1000 | ** | 16.07 | 0.00 | 1 |
| G | AMPSA | 1500 | ** | 18.94 | 0.00 | 1 |
| G | AMPSA | 2000 | ** | 18.44 | 0.00 | 1 |
| G | AMPSA | 2000 | ** | 29.03 | 0.00 | 2 |
| G | AMPSA | 2000 | ** | 10.71 | 0.00 | 3 |
| G | AMPSA | 2924 | ** | 5.13 | 28.50 | |
| G | AMPSA | 3000 | ** | 49.28 | 22.81 | 1 |
| G | AMPSA | 3000 | ** | 60.12 | 30.35 | 2 |
| G | AMPSA | 3000 | ** | 13.42 | 0.00 | 3 |
| G | AMPSA | 4000 | ** | 0.00 | 0.00 | |
| G | AMPSA | 5000 | ** | 10.38 | 0.00 | |
| G-Ag | AMPSA | 500 | <30 | 67.00 | 17.69 | 1 |
| G-Ag | AMPSA | 1000 | 54.8 | 59.78 | 0.00 | 1 |
| G-Ag | AMPSA | 1500 | 296 | 95.97 | 89.94 | 1 |
| G-Ag | AMPSA | 2000 | N/A | 93.52 | 90.13 | 1 |
| G-Ag | AMPSA | 2000 | 378 | 98.93 | 98.13 | 2 |
| G-Ag | AMPSA | 2000 | 383 | 95.02 | 87.58 | 3 |
| G-Ag | AMPSA | 2924 | 1400 | 97.42 | 98.02 | |
| G-Ag | AMPSA | 3000 | N/A | 93.40 | 89.96 | 1 |
| G-Ag | AMPSA | 3000 | 482 | 99.13 | 98.49 | 2 |
| G-Ag | AMPSA | 3000 | 150 | 98.95 | 98.52 | 3 |
| G-Ag | AMPSA | 4000 | N/A | 92.02 | 87.85 | |
| G-Ag | AMPSA | 5000 | N/A | 92.25 | 88.20 | |
| N | AMPSA | 3000 | ** | 65.00 | 56.20 | |
| N-Ag | AMPSA | 3000 | N/A | 98.51 | 98.14 | |
| G | CYST | 2000 | ** | 64.43 | 47.39 | |
| G | CYST | 3000 | ** | 61.76 | 43.45 | |
| G | CYST | 3600 | ** | 0.00 | 0.00 | |
| G | CYST | 4000 | ** | 55.57 | 34.30 | 1 |
| G | CYST | 4000 | ** | 38.34 | 0.00 | 2 |
| G | CYST | 4000 | ** | 8.37 | 0.00 | 3 |
| G | CYST | 4000 | ** | 30.90 | 9.57 | 4 |
| G | CYST | 5000 | ** | 0.00 | 0.00 | |
| G-Ag | CYST | 2000 | 324 | 90.10 | 85.35 | |
| G-Ag | CYST | 3000 | 436 | 90.33 | 85.70 | |
| G-Ag | CYST | 3600 | N/A | 92.76 | 94.54 | |
| G-Ag | CYST | 4000 | 692 | 93.81 | 90.84 | 1 |
| G-Ag | CYST | 4000 | 725 | 95.49 | 88.09 | 2 |
| G-Ag | CYST | 4000 | 750 | 92.64 | 81.65 | 3 |
| G-Ag | CYST | 4000 | 732 | 97.22 | 96.36 | 4 |
| G-Ag | CYST | 5000 | 900 | 85.62 | 78.73 | |
| N | CYST | 4000 | ** | 36.61 | 20.67 | 1 |
| N | CYST | 4000 | ** | 52.82 | 38.26 | 2 |
| N-Ag | CYST | 4000 | 744 | 94.24 | 92.79 | 1 |
| N-Ag | CYST | 4000 | 640 | 99.20 | 98.96 | 2 |
| N-Ag | CYST | 4000 | 719 | 98.43 | 97.95 | 3 |
| O | CYST | 4000 | ** | 67.04 | 56.87 | |
| O-Ag | CYST | 4000 | 778 | 97.00 | 96.07 | |
| P | CYST | 4000 | ** | 46.51 | 30.00 | |
| P-Ag | CYST | 4000 | 760 | 98.30 | 97.77 | |

N/A indicates data not available
*indicates that the lenses were prepared by the method of Example 3
**indicates that the lenses were not analyzed for silver content

### Example 3

### Preparation of Antimicrobial Contact Lenses Using Polymerizable Monomers other than PSPM and Treated with a Base Before Silver Treatment

Contact lenses prepared from a polymerizable monomer (as denoted by an asterix (*) in Table 2; in addition, Table 2 also lists the concentration used) and base monomer mix were treated with either 10% w/v solution of Na₂CO₃ in deionized water, 10% w/v solution of NaHCO₃ in deionized water, 10% w/v solution of NaOH in deionized water, or 1 M NaOMe in methanol for about 10 minutes to about 20 hours (30 lenses in 30 mL of any basic solution). The treated lenses were then removed from the basic solution and placed into deionized water (30 mL). The lenses were either rolled or stirred for a minimum of 10 minutes. This water washing procedure was repeated at least twice. The base treated lenses were then treated with a 10% w/v solution of AgNO₃ in deionized water for about 60 minutes (30 lenses in 60 mL of 10 wt. % AgNO₃ in deionized water). The base/silver treated lenses were removed from the silver solution and placed into distilled water (300 mL). The lenses were either rolled or stirred in deionized water for at least twenty minutes. This water washing procedure was repeated three (3) more times. The resulting lenses were stored in saline solution and tested to determine their antimicrobial potential.

Table 2 lists, the Base Monomer Mix (from Table 1); the polymerizable monomer of Formula I; the concentration of the polymerizable monomer of Formula I in ppm; the amount of silver incorporated into the lens; the % inhibition from the bacterial assay, using lenses made from formulation Q without any added polymerizable monomer as the control; the % inhibition from the bacterial assay using lenses made from formulation G as the control. The antimicrobial activity of formulation Q lenses and formulation G lenses are statistically the same (95% confidence (p=0.05)). In the Base Monomer Mix column, the “Ag” suffix indicates that the lenses were treated with 10% AgNO₃ as described in Example 1.

### Example 4

### Preparation of Antimicrobial Contact Lenses Containing CYST

CYST (0.2 weight percent based on weight of the monomer mix) was polymerized in monomer mix G and cured using the methods outlined in Base Monomer Formulations and Lens Preparation.

A mixture of sodium borate (3.70 g) and boric acid (18.52 g) was placed in a 2 L volumetric flask and diluted to volume with deionized water to give Borate Buffered Packing Solution. Silver nitrate (0.1042 g) was weighed into a 100 mL volumetric flask and de-ionized water was added to volume to give Silver Stock Solution. The Silver Stock Solution was further diluted with the Borate Buffered Solution to give a working solution concentration of 0.33 ug Ag/mL. The cured lenses were transferred into vials containing 3 mL of the working solution. The vials were sealed and autoclaved for 2 hours@121° C. The treated lenses were removed from the vials and washed with several portions of de-ionized water and subsequently re-packaged in Sodium Chloride Packing Solution (3 mL in vials of 0.85% sodium chloride, 0.9% boric acid, 0.18% sodium borate, 0.01% EDTA adjusted to pH of 7.3). The resulting lenses were analyzed for silver content by Instrumental Neutron Activation Analysis (INAA). Lenses produced by this method were characterized by a silver concentration of 46 ug/g.

### Example 5

### Movement of Lenses

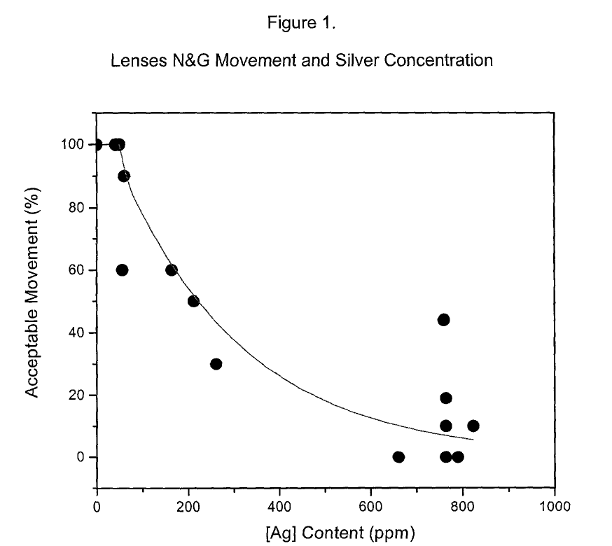

Lenses were prepared using the method of Examples 1 2, and 4. The amount of polymerizable ligands and the base monomer are listed. To determine the amount of silver (“[Ag]”) present in each lens type, samples were sent Galbraith Laboratories, Inc. (Knoxyille, Tenn.) for silver analysis by inductively coupled plasma atomic emission. The first ten lens types in Table 3 were tested on ten (10) subjects per type of lens using the push up assay (Contact Lens Practice, Chapman & Hall, 1994, edited by M. Ruben and M. Guillon, pgs. 589-99). The last six (6) entries in Table 3 were tested on twenty-three (23) subjects per type of lens using the push-up assay. All lenses were evaluated 30 minutes after placing the lenses on patients' eyes. The percentage of lenses having acceptable movement qualities was calculated as follows. Any lens having a score of greater than −2 on the push up test was an acceptable lens. In a each patient study, the number of acceptable lenses was divided by the total number lenses. Lenses having a percentage of movement equal to or greater than 50% are the preferred lenses. In addition, prior to insertions in a patient's eyes the efficacy of the lenses N and G were tested using microbial tests, A and B respectively. The activity of the lenses in these assays is listed in Table 3 as a the log reduction of the assay. FIG. 1 shows the percentage lenses having acceptable movement vs the amount of silver in each lens.

TABLE 3

| Base | Monomer of | [Formula I] | [Ag] | Log Reduction |
|---|---|---|---|---|
| Monomer Mix | Formula I | ppm | ppm | Assays |
| N-Ag | CYST | 4000 | 764 | 1.80 |
| N-Ag¹ | CYST | 4000 | 760 | 1.60 |
| N-Ag | CYST | 4000 | 823 | 1.10 |
| N-Ag | CYST | 4000 | 261 | 1.23 |
| N-Ag | CYST | 4000 | 661 | 1.48 |
| N-Ag | CYST | 4000 | 212 | 0.84 |
| N-Ag² | CYST | 4000 | 790 | 1.17 |
| G-Ag | CYST | 2000 | 60 | 1.41 |
| G-Ag | CYST | 2000 | 56 | 1.45 |
| G-Ag | CYST | 2000 | 164 | 1.24 |
| G-Ag | CYST | 2000 | 50 | 2.17 |
| G-Ag | CYST | 2000 | 41 | 2.24 |
| G-Ag | CYST | 2000 | 42 | 2.18 |
| G-Ag | CYST | 2000 | 49 | 2.01 |
| G-Ag | CYST | 2000 | 43 | 1.91 |
| G-Ag | CYST | 2000 | 49 | 1.00 |
| G-Ag | APDS | 2000 | 313 | 1.57 |

¹lenses coated with PAA
²lenses coated with pHEMA

### Example 6

### Movement of Lenses

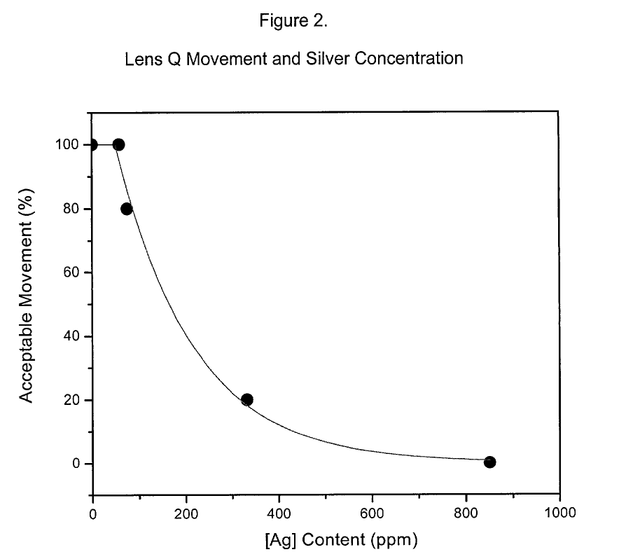

Lenses were prepared using the method of Examples 1 and 2. The amount of polymerizable ligands and the base monomer are listed. To determine the amount of silver (“[Ag]”) present in each lens type, samples were sent Galbraith Laboratories, Inc. (Knoxyille, Tenn.) for silver analysis by inductively coupled plasma atomic emission spectroscopy before the lenses were inserted into the eyes of patients. The lens types listed in Table 4 were tested on ten (10) subjects per type of lens using the push up assay (Contact Lens Practice, Chapman & Hall, 1994, edited by M. Ruben and M. Guillon, pgs. 589-99). The lenses were evaluated 30 minutes after placing the lenses on patients' eyes and the percentage of acceptable movement was calculated as described in Example 5. In addition, the lenses were analyzed for pre-wear antimicrobial efficacy using Test B. The activity of the lenses in these assays is listed in Table 4 as a the log reduction of the assay. FIG. 2 shows the percentage lenses having acceptable movement vs the amount of silver in each lens.

TABLE 4

| Base | Monomer of | [Formula I] | [Ag] | Log Reduction |
|---|---|---|---|---|
| Monomer Mix | Formula I | ppm | ppm | Assays |
| Q-Ag | CYST | 2000 | ** | N/A |
| Q-Ag | CYST | 1000 | 851 | N/A |
| Q-Ag | CYST | 500 | 332 | N/A |
| Q-Ag | CYST | 250 | 58 | 1.94 |

** indicates not analyze for silver concentration

### Example 7

### Movement of Lenses

In addition to the testing procedures of Example 5, the lenses of Example 4 were tested after 10 hours, 1 week, 2 weeks and 4 weeks of daily wear use. The percentage of acceptable movement of the lenses was 100%. The lenses were removed from patients' eyes after 10 hours, 1 week, 2 weeks and 4 weeks of use and subsequently analyzed to determine the amount of silver remaining in the lenses. Prior to use, the lenses contained 46 ppm of silver (STD 5). Forty percent (40%) of the silver was lost between 10 hours and one week of daily wear. After one week of daily wear, no further loss of silver was observed.

### Example 8

### Silver Spike Solution and Lenses

CYST (0.4 weight percent based on weight of the monomer mix) was polymerized in monomer mix N and cured using the methods outlined in Base Monomer Formulations and Lens Preparation.

Silver nitrate (0.0787 g) was weighed into a 25 mL volumetric flask and Borate Buffered Packing Solution was added to volume to give Solution C ([Ag], 2000 μg/mL). Solution C was further diluted with the Borate Buffered Solution to give the Silver Spike Solution ([Ag], 20 μg/mL). The cured lenses were transferred to vials containing Borate Buffered Packing Solution (3 mL, made by the method of Example 11) and 50 μL of Silver Spike Solution was added using an eppendorf pipet. The vials were sealed and autoclaved for 3 consecutive cycles of 30 minutes each @121° C. The resulting lenses were analyzed for silver content by Instrumental Neutron Activation Analysis (INAA). The average silver content of the lenses was 45.4 μg/g.

### Example 9

### Silver Spike Solution and Lenses

CYST (0.2 weight percent based on the weight of the monomer mix) was polymerized in monomer mix G and cured using the methods outlined in Base Monomer Formulations and Lens Preparation. A number of lenses were placed in a jacketed beaker, (60° C.) containing 800 mL of sodium borohydride solution (200 μg/mL). The lenses were agitated using a magnetic stirrer for 15 min and subsequently rinsed several times with de-ionized water at 60° C.

Subsequently, the lenses were placed in vials containing Borate Buffered Packing Solution (3 mL, made by the method of Example 11) and 50 μL of Silver Spike Solution (Example 8) was added using an eppendorf pipet. The vials were sealed and autoclaved for 3 consecutive cycles of 30 minutes each @121° C. The resulting lenses were analyzed for silver content by INAA. The average silver content of the lenses was 44.1 μg/g.

### Example 10

### Silver Spike Solution and Lenses

CYST (02 weight percent based on the weight of the monomer mix) was polymerized in monomer mix G and cured using the methods outlined in Base Monomer Formulations and Lens Preparation. A number of lenses were placed in a jacketed beaker, (60° C.) containing 800 mL of sodium borohydride solution (200 μg/mL). The lenses were agitated using a magnetic stirrer for 15 min and subsequently rinsed several times with de-ionized water at 60° C.

Subsequently, the lenses were placed in vials containing Sodium Chloride Packing Solution (3 mL, of 0.85% sodium chloride, 0.9% boric acid, 0.18% sodium borate, 0.01% EDTA adjusted to pH of 7.3) and 50 μL of Silver Spike Solution was added using an eppendorf pipet. The vials were sealed and autoclaved for 3 consecutive cycles of 30 minutes each @121° C. The resulting lenses were analyzed for silver content by INAA. The average silver content of the lenses was 44.2 μg/g.

### Example 11

### Preparation of Antimicrobial Contact Lenses Containing CYST

CYST (0.2 weight percent based on the weight of the monomer mix) was polymerized in monomer mix G and cured using the methods outlined in Base Monomer Formulations and Lens Preparation.

A mixture of sodium borate (1.85 g) and boric acid (9.26 g) was placed in a 1 L volumetric flask and diluted to volume with deionized water to give Borate Buffered Packing Solution. Silver nitrate (0.0162 g) was weighed into a 50 mL volumetric flask and de-ionized water was added to volume to give Silver Stock Solution. The Silver Stock Solution was further diluted with the Borate Buffered Solution to give Solution A (1.0 μg/mL) and Solution B (0.5 μg/mL). The cured lenses were transferred into vials containing 3 mL of either Solution A or Solution B. The vials were sealed and autoclaved for 3 consecutive cycles of 30 minutes each @121° C. The resulting lenses were analyzed for silver content by inductively coupled plasma atomic emission spectroscopy. Lenses made using Solution A had an average silver content of 151 μg/g. Lenses made using Solution B had an average silver content of 75 μg/g.

What is claimed is:

1. An antimicrobial lens comprising silver and a polymer comprising a monomer of Formula I, II, III or IV 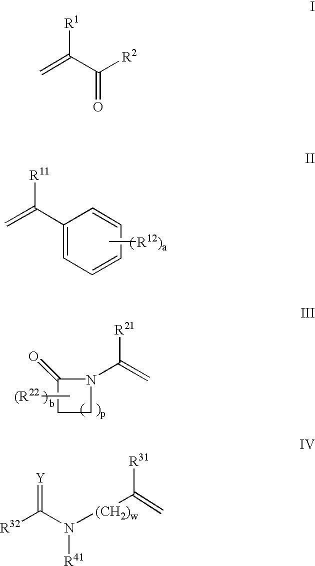
wherein
R¹ is hydrogen or C₁₋₆alkyl;
R² is —OR³, —NH—R³—S—(CH₂)_{d}—R³,or —(C H₂)_{d}—R³, wherein
d is 0-8;
R³ is substituted C₁₋₆alkyl
where the alkyl substituents are selected from one or more members of the group consisting of carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea, and substituted phenylthiourea
wherein the C₁₋₆alkyldisulfide, phenyldisulfide, C₁₋₆alkylurea, C₁₋₆alkylthiourea, phenylurea, and
phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR⁴R⁵)_{q}—(CHR⁶)ₘ—SO₃H
wherein R⁴, R⁵, and R⁶ are independently selected from the group consisting of hydrogen, halogen, hydroxyl, and C₁₋₆alkyl,
q is 1-6, and
m is 0-6;
—(CH₂)ₙ—S—S—(CH₂)ₓNH—C(O)CR⁷CH₂,
wherein R⁷ is hydrogen or C₁₋₆alkyl,
n is 1-6, and
x is 1-6;
—(CR⁸R⁹)ₜ—(CHR¹⁰)ᵤ—P(O)(OH)₂
wherein R⁸, R⁹, and R¹⁰ are independently selected from the group consisting of hydrogen, halogen, hydroxyl, and C₁₋₆alkyl,
t is 1-6, and
u is 0-6;
phenyl;
benzyl;
pyridinyl;
pyrimidinyl;
pyrazinyl;
benzimidazolyl;
benzothiazolyl;
benzotriazolyl;
naphthaloyl;
quinolinyl;
indolyl;
thiadiazolyl;
triazolyl;
4-methylpiperidin-1-yl;
4-methylpiperazin-1-yl;
substituted phenyl;
substituted benzyl;
substituted pyridinyl;
substituted pyrimidinyl;
substituted pyrazinyl;
substituted benzimidazolyl;
substituted benzothiazolyl;
substituted benzotriazolyl;
substituted naphthaloyl;
substituted quinolinyl;
substituted indolyl;
substituted thiadiazolyl;
substituted triazolyl;
substituted 4-methylpiperidin-1-yl; or
substituted 4-methylpiperazin-1-yl,
wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methyl piperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl) phosphonyl, N-(amino-4-methylpiperazinyl) phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
a is 1-5;
R¹¹ is hydrogen or C₁₋₆alkyl;
R¹² is hydroxyl, sulfonic acid, phosphonic acid, carboxylic acid, acetamide, thioC₁₋₆alkylcarbonyl, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, —OR¹³, —NH—R¹³—S—(CH₂)_{d}—R¹³, —(CH₂)_{d}—R³, —C(O)NH—(CH₂)_{d}—R¹³, —C(O)—(CH₂)_{d}—R³, substituted C₁₋₆alkyldisulfide, substituted phenyldisulfide, substituted C₁₋₆alkylurea, substituted phenylurea, substituted phenylthiourea or substituted C₁₋₆alkylthiourea wherein the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
where
d is 0-8;
R¹³ is thioC₁₋₆alkylcarbonyl;
substituted C₁₋₆alkyl
where the alkyl substituents are selected from one or more members of the group consisting of hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR¹⁴R¹⁵)_{q}—(CHR¹⁶)ₘ—SO₃H
where R¹⁴, R¹⁵, and R¹⁶ are independently selected from the group consisting of hydrogen, halogen, hydroxyl, and C₁₋₆alkyl,
q is 1-6, and
m is 0-6;
—(CH₂)ₙ—S—S—(CH₂)ₓNH—C(O)CR¹⁷CH₂,
where R¹⁷ is hydrogen or C₁₋₆alkyl,
n is 1-6, and
x is 1-6;
—(CR¹⁸R¹⁹)ₜ—(CHR²⁰)ᵤ—P(O)(OH)₂
where R¹⁸, R¹⁹, and R²⁰ are independently selected from the group consisting of hydrogen, halogen, hydroxyl, and C₁₋₆alkyl,
t is 1-6, and
u is 0-6;
phenyl;
benzyl;
pyridinyl;
pyrimidinyl;
pyrazinyl;
benzimidazolyl;
benzothiazolyl;
benzotriazolyl;
naphthaloyl;
quinolinyl;
indolyl;
thiadiazolyl;
triazolyl;
4-methylpiperidin-1-yl;
4-methylpiperazin-1-yl;
substituted phenyl;
substituted benzyl;
substituted pyridinyl;
substituted pyrimidinyl;
substituted pyrazinyl;
substituted benzimidazolyl;
substituted benzothiazolyl;
substituted benzotriazolyl;
substituted naphthaloyl;
substituted quinolinyl;
substituted indolyl;
substituted thiadiazolyl;
substituted triazolyl;
substituted 4-methylpiperidin-1-yl; or
substituted 4-methylpiperazin-1-yl
wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, 57 N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methylpiperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl) phosphonyl, N-(amino-4-methylpiperazinyl) phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
b is 1-5;
p is 1-5;
R²¹ is hydrogen;
R²² is hydroxyl, sulfonic acid, phosphonic acid, carboxylic acid, thioC₁₋₆alkylcarbonyl, thioC₁₋₆alkylaminocarbonyl, C₁₋₆alkyldisulfide, phenyldisulfide, —C(O)NH(CH₂)₁₋₆—SO₃H, —C(O)NH(CH₂)₁₋₆—P(O)(OH)₂, —OR²³, —NH—R²³—C(O)NH—(CH₂)_{d}—R²³—S—(CH₂)_{d}—R²³, —(CH₂)_{d}—R²³, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted, C₁₋₆alkylthiourea substituted phenylurea or substituted phenylthiourea wherein the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile,
where
d is 0-8;
R²³ is thioC₁₋₆alkylcarbonyl,
C₁₋₆alkyl,
substituted C₁₋₆alkyl
where the alkyl substituents are selected from one or more members of the group consisting of C₁₋₆alkyl, halo C₁₋₆alkyl, halogen, hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR²⁴R²⁵)_{q}—(CHR²⁶)ₘ—SO₃H
where R²⁴, R²⁵, and R²⁶ are independently selected from the group consisting of hydrogen, halogen, hydroxyl, and C₁₋₆alkyl,
q is 1-6, and
m is 0-6
—(CH₂)ₙ—S—S—(CH₂)ₓNH—C(O)CR²⁷CH₂,
where R²⁷ is hydrogen or C₁₋₆alkyl,
n is 1-6, and
x is 1-6;
—(CR²⁸R²⁹)ₜ—(CHR³⁰)ᵤ—P(O)(OH)₂
where R²⁸, R²⁹, and R³⁰ are independently selected from the group consisting of hydrogen, halogen, hydroxyl, and C₁₋₆alkyl,
t is 1-6, and
u is 0-6;
phenyl;
benzyl;
pyridinyl;
pyrimidinyl;
pyrazinyl;
benzimidazolyl;
benzothiazolyl;
benzotriazolyl;
naphthaloyl;
quinolinyl;
indolyl;
thiadiazolyl;
triazolyl;
4-methylpiperidin-1-yl;
4-methylpiperazin-1-yl;
substituted phenyl;
substituted benzyl;
substituted pyridinyl;
substituted pyrimidinyl;
substituted pyrazinyl;
substituted benzimidazolyl;
substituted benzothiazolyl;
substituted benzotriazolyl;
substituted naphthaloyl;
substituted quinolinyl;
substituted indolyl;
substituted thiadiazolyl;
substituted triazolyl;
substituted 4-methylpiperidin-1-yl; or
substituted 4-methylpiperazin-1-yl,
wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methylpiperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl) phosphonyl, N-(amino-4-methylpiperazinyl) phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
w is 0-1;
Y is oxygen or sulfur;
R³¹ is hydrogen or C₁₋₆alkyl;
R³² is hydroxyl, sulfonic acid, phosphonic acid, carboxylic acid, thioC₁₋₆alkylcarbonyl, thioC₁₋₆alkylaminocarbonyl, —C(O)NH—(CH₂)_{d}—R³³, —O—R³³, —NH—R³³—S—(CH₂)_{d}—R³³, —(CH₂)_{d}—R³³, C₁₋₆alkyldisulfide, phenyldisulfide, urea, C₁₋₆alkylurea, phenylurea, thiourea, C₁₋₆alkylthiourea, phenylthiourea, C₁₋₆alkylamine, phenylamine, substituted C₁₋₆alkyldisulfide, substituted phenyldisulfide, substituted phenylurea, substituted C₁₋₆alkylamine, substituted phenylamine, substituted phenylthiourea, substituted C₁₋₆alkylurea or substituted C₁₋₆alkylthiourea wherein the substitutents are selected from the group consisting of C₁₋₆alkyl, haloC₁₋₆alkyl, halogen, hydroxyl, carboxylic acid, sulfonic acid, phosphonic acid, amine, amidine, acetamide, and nitrile
where
d is 0-8;
R³³ is thioC₁₋₆alkylcarbonyl,
C₁₋₆alkyl,
substituted C₁₋₆alkyl
where the alkyl substituents are selected from one or more members of the group consisting of C₁₋₆alkyl, halo C₁₋₆alkyl, halogen, hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea or substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR³⁴R³⁵)_{q}—(CHR³⁶)ₘ—SO₃H
where R³⁴, R³⁵, and R³⁶ are independently selected from the group consisting of hydrogen, halogen, hydroxyl, and C₁₋₆alkyl,
q is 1-6, and
m is 0-6;
—(CH₂)ₙ—S—S—(CH₂)ₓNH—C(O)CR³⁷CH₂,
where R³⁷ is hydrogen or C₁₋₆alkyl,
n is 1-6, and
x is 1-6;
—(CR³⁸R³⁹)ₜ—(CHR⁴⁰)ᵤ—P(O)(OH)₂
where R³⁸, R³⁹, and R⁴⁰ are independently selected from the group consisting of hydrogen, halogen, hydroxyl, and C₁₋₆alkyl,
t is 1-6, and
u is 0-6;
phenyl;
benzyl;
pyridinyl;
pyrimidinyl;
pyrazinyl;
benzimidazolyl;
benzothiazolyl;
benzotriazolyl;
naphthaloyl;
quinolinyl;
indolyl;
thiadiazolyl;
triazolyl;
4-methylpiperidin-1-yl;
4-methylpiperazin-1-yl;
substituted phenyl;
substituted benzyl;
substituted pyridinyl;
substituted pyrimidinyl;
substituted pyrazinyl;
substituted benzimidazolyl;
substituted benzothiazolyl;
substituted benzotriazolyl;
substituted naphthaloyl;
substituted quinolinyl;
substituted indolyl;
substituted thiadiazolyl;
substituted triazolyl;
substituted 4-methylpiperidin-yl; or
substituted 4-methylpiperazin-1-yl,
wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methylpiperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl) phosphonyl, N-(amino-4-methylpiperazinyl) phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
R⁴¹ is hydrogen, C₁₋₆alkyl, phenyl, C₁₋₆alkylcarbonyl, phenylcarbonyl, substituted C₁₋₆alkyl, substituted phenyl, substituted C₁₋₆alkylcarbonyl or substituted phenylcarbonyl,
wherein
the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile.

2. The antimicrobial lens of claim 1 comprising a polymer comprising a monomer of Formula I.

3. The antimicrobial lens of claim 2 wherein,
R¹ is hydrogen or C₁₋₃alkyl;
R² is NH—R³;
d is 0
R³ is substituted phenyl, —(CR⁴R⁵)_{q}—(CHR⁶)ₘ—SO₃H,
—(CR⁸R⁹)ₜ—(CHR¹⁰)ᵤ—P(O)(OH)₂ or —(CH₂)ₙ—S—S—(CH₂)ₓNH—C(O)CR⁷CH₂;
R⁴ is hydrogen or C₁₋₃alkyl;
R⁵ is hydrogen or C₁₋₃alkyl;
R⁶ is hydrogen or C₁₋₃alkyl;
q is 1-3;
m is 1-3;
R⁷ is hydrogen or C₁₋₃alkyl;
R⁸ is hydrogen or C₁₋₃alkyl;
R⁹ is hydrogen or C₁₋₃alkyl;
R¹⁰ is hydrogen or C₁₋₃alkyl;
t is 1-3;
u is 1-3;
n is 2-4; and
x is 2-4.

4. The antimicrobial lens of claim 2 wherein the lens is a soft contact lens.

5. The antimicrobial lens of claim 2 wherein the monomer of Formula I is present at about 0.01 to about 1.5 weight percent.

6. The antimicrobial lens of claim 2 wherein the monomer of Formula I is present at about 0.01 to about 0.8 weight percent.

7. The antimicrobial lens of claim 2 wherein the monomer of Formula I is present at about 0.01 to about 0.3 weight percent.

8. The antimicrobial lens of claim 2 wherein the monomer of Formula I is present at about 0.01 to about 0.2 weight percent.

9. The antimicrobial lens of claim 2 wherein the monomer of Formula I is present at about 0.01 to about 0.09 weight percent.

10. The antimicrobial lens of claim 2 wherein the lens is a silicone hydrogel.

11. The antimicrobial lens of claim 2 wherein, the lens is etafilcon A, balafilcon, A, acquafilcon A, lenefilcon A, or lotrafilcon A.

12. The antimicrobial lens of claim 2 wherein,
R¹ is hydrogen or methyl;
R² is NH—R³;
R³ is —(CR⁴R⁵)_{q}—(CHR⁶)ₘ—SO₃H, —(CR⁸R⁹)ₜ—(CHR¹⁰)ᵤ—P(O)(OH)₂ or —(CH₂)ₙ—S—S—(CH₂)ₓNH—C(O)CHR⁷CH₂;
R⁴ is hydrogen or methyl;
R⁵ is hydrogen or methyl;
q is 1-2;
m is 1-2;
R⁶ is hydrogen or methyl;
R⁷ is hydrogen;
R⁸ is hydrogen or methyl;
R⁹ is hydrogen or methyl;
R¹⁰ is hydrogen or methyl;
t is 1;
u is 1-2;
n is 2-3; and
x is 2-3.

13. The antimicrobial lens of claim 2 wherein the monomer of Formula I is selected from the group consisting of

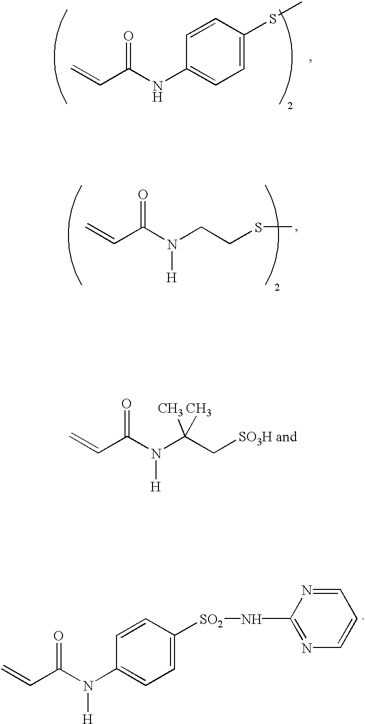

14. The antimicrobial lens of claim 2 wherein silver is present at about 20 ppm to about 1,200 ppm.

15. The antimicrobial lens of claim 2 wherein silver is present at about 20 ppm to about 600 ppm.

16. The antimicrobial lens of claim 2 wherein silver is present at about 20 ppm to about 150 ppm.

17. The antimicrobial lens of claim 2 wherein silver is present at about 20 ppm to about 75 ppm.

18. The antimicrobial lens of claim 2 wherein the lens is a silicone hydrogel and the monomer of Formula I is

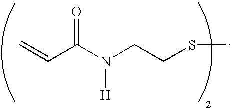

19. The antimicrobial lens of claim 18 wherein silver is present at about 20 ppm to about 150 ppm and the monomer of Formula I is present at about 0.01 to about 1.5 weight percent.

20. The antimicrobial lens of claim 2 wherein the lens is etafilcon A, balafilcon, A, acquafilcon A, lenefilcon, or lotrafilcon A and the monomer of Formula I is

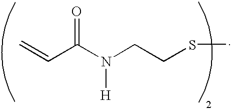

21. The antimicrobial lens of claim 20 wherein silver is present at about 20 ppm to about 150 ppm and the monomer of Formula I is present at about 0.01 to about 1.5 weight percent.

22. The antimicrobial lens of claim 21 wherein the lens is etafilcon A.

23. The antimicrobial lens of claim 21 wherein the lens is acquafilcon A.

24. The lens of claim 23 wherein silver is present at about 20 ppm to about 75 ppm.

25. The antimicrobial lens of claim 1 comprising a polymer comprising a monomer of Formula II.

26. The antimicrobial lens of claim 25 wherein,
a is 1-2,
R¹¹ is hydrogen or C₁₋₃alkyl,
R¹² is sulfonic acid, carboxylic acid, phosphonic acid, C₁₋₆alkyldisulfide, C₁₋₆alkylsulfide, phenyldisulfide, substiuted phenyldisulfide or NH—R¹³,
R¹³ is thioC₁₋₆alkylcarbonyl.

27. The antimicrobial lens of claim 25 wherein the monomer of Formula II is selected from the group consisting of

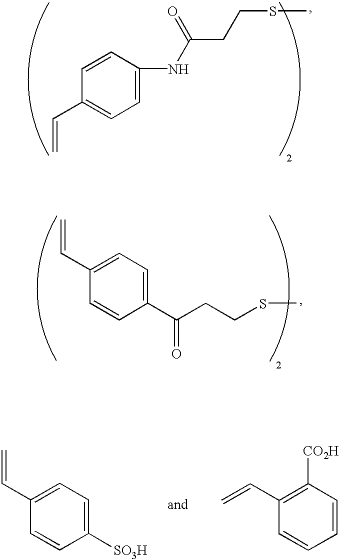

28. The antimicrobial lens of claim 25 wherein the lens is a soft contact lens.

29. The antimicrobial lens of claim 25 wherein the monomer of Formula II is present at about 0.01 to about 1.5 weight percent.

30. The antimicrobial lens of claim 25 wherein the monomer of Formula II is present at about 0.01 to about 0.8 weight percent.

31. The antimicrobial lens of claim 25 wherein the monomer of Formula II is present at about 0.01 to about 0.3 weight percent.

32. The antimicrobial lens of claim 25 wherein the lens is etafilcon A, balafilcon, A, acquafilcon A, lenefilcon A, or lotrafilcon A.

33. The antimicrobial lens of claim 25 wherein silver is present at about 20 ppm to about 150 ppm and the monomer of Formula II is present at about 0.01 to about 1.5 weight percent.

34. The antimicrobial lens of claim 33 wherein the lens is etafilcon A or acquafilcon A.

35. The antimicrobial lens of claim 1 comprising a polymer comprising a monomer of Formula III.

36. The antimicrobial lens of claim 35 wherein,
p is 1-3;
b is 1-2;
R²¹ is hydrogen;
R²² is sulfonic acid, phosphonic acid, carboxylic acid, thioC₁₋₆alkylcarbonyl, thioC₁₋₆alkylaminocarbonyl, C₁₋₆alkyldisulfide, C₁₋₆alkylsulfide, phenyldisulfide, substiuted phenyldisulfide,
H₃OS—(CH₂)₁₋₆NHC(O) or
(HO)₂(O)P—(CH₂)₁₋₆NHC(O)—.

37. The antimicrobial lens of claim 35 wherein the monomer of Formula III is selected from the group consisting of

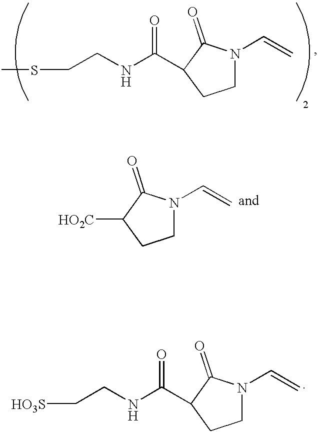

38. The antimicrobial lens of claim 35 wherein the lens is a soft contact lens.

39. The antimicrobial lens of claim 35 wherein the monomer of Formula III is present at about 0.01 to about 1.5 weight percent.

40. The antimicrobial lens of claim 35 wherein the monomer of Formula III is present at about 0.01 to about 0.8 weight percent.

41. The antimicrobial lens of claim 35 wherein the monomer of Formula III is present at about 0.01 to about 0.3 weight percent.

42. The antimicrobial lens of claim 35 wherein, the lens is etafilcon A, balafilcon, A, acquafilcon A, lenefilcon A, or lotrafilcon A.

43. The antimicrobial lens of claim 35 wherein silver is present at about 20 ppm to about 150 ppm and the monomer of Formula III is present at about 0.01 to about 1.5 weight percent.

44. The antimicrobial lens of claim 43 wherein the lens is etafilcon A or acquafilcon A.

45. The antimicrobial lens of claim 1 comprising a polymer comprising a monomer of Formula IV.

46. The antimicrobial lens of claim 45 wherein,
w is 0-1;
R³¹ is hydrogen;
R³² is amine, C₁₋₃alkylamine, phenylamine, substituted phenylamine;
thioC₁₋₃alkylcarbonyl;
R⁴¹ is hydrogen.

47. The antimicrobial lens of claim 45 wherein the monomer of Formula IV is selected from the group consisting of

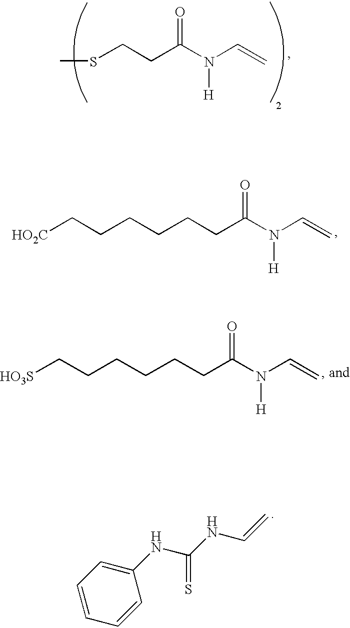

48. The antimicrobial lens of claim 45 wherein the lens is a soft contact lens.

49. The antimicrobial lens of claim 45 wherein the monomer of Formula IV is present at about 0.01 to about 1.5 weight percent.

50. The antimicrobial lens of claim 45 wherein the monomer of Formula IV is present at about 0.01 to about 0.8 weight percent.

51. The antimicrobial lens of claim 45 wherein the monomer of Formula IV is present at about 0.01 to about 0.3 weight percent.

52. The antimicrobial lens of claim 45 wherein the lens is etafilcon A, balafilcon, A, acquafilcon A, lenefilcon A, or lotrafilcon A.

53. The antimicrobial lens of claim 45 wherein silver is present at about 20 ppm to about 150 ppm and the monomer of Formula IV is present at about 0.01 to about 1.5 weight percent.

54. The antimicrobial lens of claim 53 wherein the lens is etafilcon A or acquafilcon A.

55. A method of producing an antimicrobial lens comprising, silver and a polymer comprising a monomer of Formula I, II, III or IV 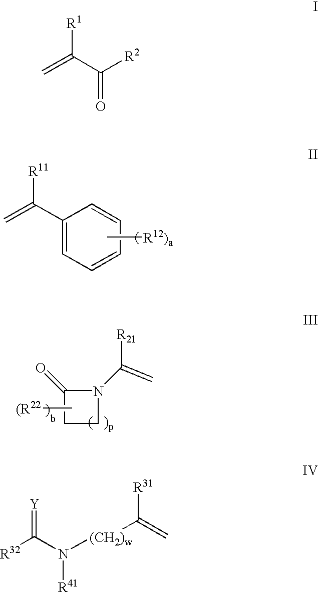
wherein
R¹ is hydrogen or C₁₋₆alkyl;
R² is —OR³, —NH—R³, —S—(CH₂)_{d}—R³,or —(CH₂)_{d}—R³, wherein
d is 0-8;
R³ is substituted C₁₋₆alkyl
where the alkyl substituents are selected from one or more members of the group consisting of carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
(CR⁴R⁵)_{q}—(CHR⁶)ₘ—SO₃H
wherein R⁴, R⁵, and R⁶ are independently selected from the group consisting of hydrogen, halogen, hydroxyl, and C₁₋₆alkyl,
q is 1-6, and
m is 0-6;
—(CH₂)ₙ—S—S—(CH₂)ₓNH—C(O)CR⁷CH₂,
wherein R⁷ is hydrogen or C₁₋₆alkyl,
n is 1-6, and
x is 1-6;
—(CR⁸R⁹)ₜ—(CHR¹⁰)ᵤ—P(O)(OH)₂
wherein R⁸, R⁹, and R¹⁰ are independently selected from the group consisting of hydrogen, halogen, hydroxyl, and C₁₋₆alkyl,
t is 1-6, and
u is 0-6;
phenyl;
benzyl;
pyridinyl;
pyrimidinyl;
pyrazinyl;
benzimidazolyl;
benzothiazolyl;
benzotriazolyl;
naphthaloyl;
quinolinyl;
indolyl;
thiadiazolyl;
triazolyl;
4-methylpiperidin-1-yl;
4-methylpiperazin-1-yl;
substituted phenyl;
substituted benzyl;
substituted pyridinyl;
substituted pyrimidinyl;
substituted pyrazinyl;
substituted benzimidazolyl;
substituted benzothiazolyl;
substituted benzotriazolyl;
substituted naphthaloyl;
substituted quinolinyl;
substituted indolyl;
substituted thiadiazolyl;
substituted triazolyl;
substituted 4-methylpiperidin-1-yl; or
substituted 4-methylpiperazin-1-yl,
wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methylpiperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl) phosphonyl, N-(amino-4-methylpiperazinyl) phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
a is 1-5;
R¹¹ is hydrogen or C₁₋₆alkyl;
R¹² is hydroxyl, sulfonic acid, phosphonic acid, carboxylic acid, acetamide, thioC₁₋₆alkylcarbonyl, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, —OR¹³, —NH—R¹³—S—(CH₂)_{d}—R¹³, —(CH₂)_{d}—R¹³, —C(O)NH—(CH₂)_{d}—R¹³—C(O)—(CH₂)_{d}—R¹³, substituted C₁₋₆alkyldisulfide, substituted phenyldisulfide, substituted C₁₋₆alkylurea, substituted phenylurea, substituted phenylthiourea or substituted C₁₋₆alkylthiourea wherein the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
where
d is 0-8;
R¹³ is thioC₁₋₆alkylcarbonyl;
substituted C₁₋₆alkyl
where the alkyl substituents are selected from one or more members of the group consisting of hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR¹⁴R¹⁵)_{q}—(CHR¹⁶)ₘ—SO₃H
where R¹⁴, R¹⁵, and R¹⁶ are independently selected from the group consisting of hydrogen, halogen, hydroxyl, and C₁₋₆alkyl,
q is 1-6, and
m is 0-6;
—(CH₂)ₙ—S—S—(CH₂)ₓNH—C(O)CR¹⁷CH₂,
where R¹⁷ is hydrogen or C₁₋₆alkyl,
n is 1-6, and
x is 1-6;
—(CR¹⁸R¹⁹)ₜ—(CHR²⁰)ᵤ—P(O)(OH)₂
where R¹⁸, R¹⁹, and R²⁰ are independently selected from the group consisting of hydrogen, halogen, hydroxyl, and C₁₋₆alkyl,
t is 1-6, and
u is 0-6;
phenyl;
benzyl;
pyridinyl;
pyrimidinyl;
pyrazinyl;
benzimidazolyl;
benzothiazolyl;
benzotriazolyl;
naphthaloyl;
quinolinyl;
indolyl;
thiadiazolyl;
triazolyl;
4-methylpiperidin-1-yl;
4-methylpiperazin-1-yl;
substituted phenyl;
substituted benzyl;
substituted pyridinyl;
substituted pyrimidinyl;
substituted pyrazinyl;
substituted benzimidazolyl;
substituted benzothiazolyl;
substituted benzotriazolyl;
substituted naphthaloyl;
substituted quinolinyl;
substituted indolyl;
substituted thiadiazolyl;
substituted triazolyl;
substituted 4-methylpiperidin-1-yl; or
substituted 4-methylpiperazin-1-yl
wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methylpiperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl) phosphonyl, N-(amino-4-methylpiperazinyl) phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
b is 1-5;
p is 1-5;
R²¹ is hydrogen;
R²² is hydroxyl, sulfonic acid, phosphonic acid, carboxylic acid, thioC₁₋₆alkylcarbonyl, thioC₁₋₆alkylaminocarbonyl, C₁₋₆alkyldisulfide, phenyldisulfide, —C(O)NH(CH₂)₁₋₆—SO₃H, —C(O)NH(CH₂)₁₋₆—P(O)(OH)₂, —OR²³, —NH—R²³—C(O)NH—(CH₂)_{d}—R²³—S—(CH₂)_{d}—R²³, —(CH₂)_{d}—R²³, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted, C₁₋₆alkylthiourea substituted phenylurea or substituted phenylthiourea wherein the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile,
where
d is 0-8;
R²³ is thioC₁₋₆alkylcarbonyl,
C₁₋₆alkyl,
substituted C₁₋₆alkyl
where the alkyl substituents are selected from one or more members of the group consisting of C₁₋₆alkyl, halo C₁₋₆alkyl, halogen, hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR²⁴ R²⁵)_{q}—(CHR²⁶)ₘ—SO₃H
where R²⁴, R²⁵, and R²⁶ are independently selected from the group consisting of hydrogen, halogen, hydroxyl, and C₁₋₆alkyl,
q is 1-6, and
m is 0-6
—(CH₂)ₙ—S—S—(CH₂)ₓNH—C(O)CR²⁷CH₂,
where R²⁷ is hydrogen or C₁₋₆alkyl,
n is 1-6, and
x is 1-6;
—(CR²⁸R²⁹)ₜ—(CHR³⁰)ᵤ—P(O)(OH)₂
where R²⁸, R²⁹, and R³⁰ are independently selected from the group consisting of hydrogen, halogen, hydroxyl, and C₁₋₆alkyl,
t is 1-6, and
u is 0-6;
phenyl;
benzyl;
pyridinyl;
pyrimidinyl;
pyrazinyl;
benzimidazolyl;
benzothiazolyl;
benzotriazolyl;
naphthaloyl;
quinolinyl;
indolyl;
thiadiazolyl;
triazolyl;
4-methylpiperidin-1-yl;
4-methylpiperazin-1-yl;
substituted phenyl;
substituted benzyl;
substituted pyridinyl;
substituted pyrimidinyl;
substituted pyrazinyl;
substituted benzimidazolyl;
substituted benzothiazolyl;
substituted benzotriazolyl;
substituted naphthaloyl;
substituted quinolinyl;
substituted indolyl;
substituted thiadiazolyl;
substituted triazolyl;
substituted 4-methylpiperidin-1-yl; or
substituted 4-methylpiperazin-1-yl,
wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methylpiperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl) phosphonyl, N-(amino-4-methylpiperazinyl) phosphonyl, acetamide, nitrile, thiol, C₁₋₁₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
w is 0-1;
Y is oxygen or sulfur;
R³¹ is hydrogen or C₁₋₆alkyl;
R³² is hydroxyl, sulfonic acid, phosphonic acid, carboxylic acid, thioC₁₋₆alkylcarbonyl, thioC₁₋₆alkylaminocarbonyl, —C(O)NH—(CH₂)_{d}—R³³, —O—R³³, —NH—R³³—S—(CH₂)_{d}—R³³, —(CH₂)_{d}—R³³, C₁₋₆alkyldisulfide, phenyldisulfide, urea, C₁₋₆alkylurea, phenylurea, thiourea, C₁₋₆alkylthiourea, phenylthiourea, C₁₋₆alkylamine, phenylamine, substituted C₁₋₆alkyldisulfide, substituted phenyldisulfide, substituted phenylurea, substituted C₁₋₆alkylamine, substituted phenylamine, substituted phenylthiourea, substituted C₁₋₆alkylurea or substituted C₁₋₆alkylthiourea wherein the substitutents are selected from the group consisting of C₁₋₆alkyl, haloC₁₋₆alkyl, halogen, hydroxyl, carboxylic acid, sulfonic acid, phosphonic acid, amine, amidine, acetamide, and nitrile
where
d is 0-8;
R³³ is thioC₁₋₆alkylcarbonyl,
C₁₋₆alkyl,
substituted C₁₋₆alkyl
where the alkyl substituents are selected from one or more members of the group consisting of C₁₋₆alkyl, halo C₁₋₆alkyl, halogen, hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea or substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR³⁴R³⁵)_{q}—(CHR³⁶)ₘ—SO₃H
where R³⁴, R³⁵, and R³⁶ are independently selected from the group consisting of hydrogen, halogen, hydroxyl, and C₁₋₆alkyl,
q is 1-6, and
m is 0-6;
—(CH₂)ₙ—S—S—(CH₂)ₓNH—C(O)CR³⁷CH₂,
where R³⁷ is hydrogen or C₁₋₆alkyl,
n is 1-6, and
x is 1-6;
—(CR³⁸R³⁹)ₜ—(CHR⁴⁰)ᵤ—P(O)(OH)₂
where R³⁸, R³⁹, and R⁴⁰ are independently selected from the group consisting of hydrogen, halogen, hydroxyl, and C₁₋₆alkyl,
t is 1-6, and
u is 0-6;
phenyl;
benzyl;
pyridinyl;
pyrimidinyl;
pyrazinyl;
benzimidazolyl;
benzothiazolyl;
benzotriazolyl;
naphthaloyl;
quinolinyl;
indolyl;
thiadiazolyl;
triazolyl;
4-methylpiperidin-1-yl;
4-methylpiperazin-1-yl;
substituted phenyl;
substituted benzyl;
substituted pyridinyl;
substituted pyrimidinyl;
substituted pyrazinyl;
substituted benzimidazolyl;
substituted benzothiazolyl;
substituted benzotriazolyl;
substituted naphthaloyl;
substituted quinolinyl;
substituted indolyl;
substituted thiadiazolyl;
substituted triazolyl;
substituted 4-methylpiperidin-1-yl; or
substituted 4-methylpiperazin-1-yl,
wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methyl piperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methylpiperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl) phosphonyl, N-(amino-4-methylpiperazinyl) phosphonyl, acetamide, nitrile, thiol, C₁₋₆alkyidisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
R⁴¹ is hydrogen, C₁₋₆alkyl, phenyl, C₁₋₆alkylcarbonyl, phenylcarbonyl, substituted C₁₋₆alkyl, substituted phenyl, substituted C₁₋₆alkylcarbonyl or substituted phenylcarbonyl,
wherein
the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile.
where the method comprises the steps of
(a) preparing a lens comprising a monomer of Formula I, II, III or IV and
(b) treating said lens with a silver solution.

56. The method of claim 55 wherein the silver solution is aqueous silver nitrate having a concentration of about 0.1 μg/mL to about 0.3 g/mL.

57. The method of claim 55 wherein, treating comprises soaking the lens comprising a polymer of a monomer of Formula I, II, III or IV with a silver solution.

58. The method of claim 55 wherein, the lens comprising a polymer of a monomer of Formula I, II, III or IV is soaking for about 2 minutes to about 2 hours.

59. The method of claim 55 wherein, treating comprises storing the lens comprising a polymer of a monomer of Formula I, II, III or IV with a silver solution for about 20 minutes to about 5 years.

60. An antimicrobial lens comprising silver and a polymer comprising a binding monomer wherein said antimicrobial lens can reversibly bind silver.

61. The antimicrobial lens of claim 60 wherein the binding monomer has a stability constant of about 2 to about 7.3.

62. A lens case comprising silver and a polymer comprising a monomer of Formula I, II, III or IV 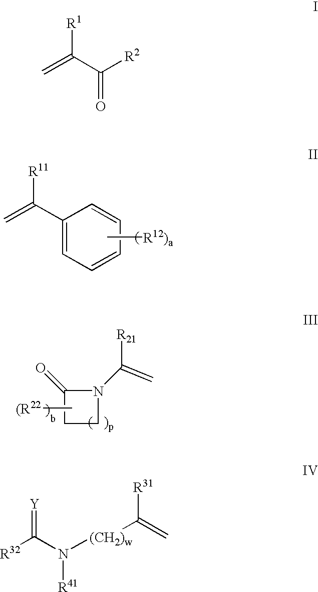
wherein
R¹ is hydrogen or C₁₋₆alkyl;
R² is —OR³, —NH—R³—S—(CH₂)_{d}—R³,or —(CH₂)_{d}—R³, wherein
d is 0-8;
R³ is substituted C₁₋₆alkyl
where the alkyl substituents are selected from one or more members of the group consisting of carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR⁴R⁵)_{q}—(CHR⁶)ₘ—SO₃H
wherein R⁴, R⁵, and R⁶ are independently selected from the group consisting of hydrogen, halogen, hydroxyl, and C₁₋₆alkyl,
q is 1-6, and
m is 0-6;
—(CH₂)ₙ—S—S—(CH₂)ₓNH—C(O)CR⁷CH₂,
wherein R⁷ is hydrogen or C₁₋₆alkyl,
n is 1-6, and
x is 1-6;
—(CR⁸R⁹)ₜ—(CHR¹⁰)ᵤ—P(O)(OH)₂
wherein R⁸, R⁹, and R¹⁰ are independently selected from the group consisting of hydrogen, halogen, hydroxyl, and C₁₋₆alkyl,
t is 1-6, and
u is 0-6;
phenyl;
benzyl;
pyridinyl;
pyrimidinyl;
pyrazinyl;
benzimidazolyl;
benzothiazolyl;
benzotriazolyl;
naphthaloyl;
quinolinyl;
indolyl;
thiadiazolyl;
triazolyl;
4-methylpiperidin-1-yl;
4-methylpiperazin-1-yl;
substituted phenyl;
substituted benzyl;
substituted pyridinyl;
substituted pyrimidinyl;
substituted pyrazinyl;
substituted benzimidazolyl;
substituted benzothiazolyl;
substituted benzotriazolyl;
substituted naphthaloyl;
substituted quinolinyl;
substituted indolyl;
substituted thiadiazolyl;
substituted triazolyl;
substituted 4-methylpiperidin-1-yl; or
substituted 4-methylpiperazin-1-yl,
wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methylpiperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl) phosphonyl, N-(amino-4-methylpiperazinyl) phosphonyl, acetamide, nitrile, thiol, C₁₋₆alkyldisulfide, C₁₋₆alkylsulfide, phenyl disulfide, urea, C_{1*6}alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
a is 1-5;
R¹¹ is hydrogen or C₁₋₆alkyl;
R¹² is hydroxyl, sulfonic acid, phosphonic acid, carboxylic acid, acetamide, thioC₁₋₆alkylcarbonyl, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, —OR¹³, —NH—R³, —S—(CH₂)_{d}—R¹³, —(CH₂)_{d}—R³, —C(O)NH—(CH₂)_{d}—R¹³—C(O)—(CH₂)_{d}—R¹³, substituted C₁₋₆alkyldisulfide, substituted phenyldisulfide, substituted C₁₋₆alkylurea, substituted phenylurea, substituted phenylthiourea or substituted C₁₋₆alkylthiourea wherein the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
where
d is 0-8;
R¹³ is thioC₁₋₆alkylcarbonyl;
substituted C₁₋₆alkyl
where the alkyl substituents are selected from one or more members of the group consisting of hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR¹⁴R¹⁵)_{q}—(CHR¹⁶)ₘ—SO₃H
where R¹⁴, R¹⁵, and R¹⁶ are independently selected from the group consisting of hydrogen, halogen, hydroxyl, and C₁₋₆alkyl,
q is 1-6, and
m is 0-6;
—(CH₂)ₙ—S—S—(CH₂)ₓNH—C(O)CR¹⁷CH₂,
where R¹⁷ is hydrogen or C₁₋₆alkyl,
n is 1-6, and
x is 1-6;
—(CR¹⁸R¹⁹)ₜ—(CHR²⁰)ᵤ—P(O)(OH)₂
where R¹⁸, R¹⁹, and R²⁰ are independently selected from the group consisting of hydrogen, halogen, hydroxyl, and C₁₋₆alkyl,
t is 1-6, and
u is 0-6;
phenyl;
benzyl;
pyridinyl;
pyrimidinyl;
pyrazinyl;
benzimidazolyl;
benzothiazolyl;
benzotriazolyl;
naphthaloyl;
quinolinyl;
indolyl;
thiadiazolyl;
triazolyl;
4-methylpiperidin-1-yl;
4-methylpiperazin-1-yl;
substituted phenyl;
substituted benzyl;
substituted pyridinyl;
substituted pyrimidinyl;
substituted pyrazinyl;
substituted benzimidazolyl;
substituted benzothiazolyl;
substituted benzotriazolyl;
substituted naphthaloyl;
substituted quinolinyl;
substituted indolyl;
substituted thiadiazolyl;
substituted triazolyl;
substituted 4-methylpiperidin-1-yl; or
substituted 4-methylpiperazin-1-yl
wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methylpiperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl) phosphonyl, N-(amino-4-methylpiperazinyl) phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyidisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
b is 1-5;
p is 1-5;
R²¹ is hydrogen;
R²² is hydroxyl, sulfonic acid, phosphonic acid, carboxylic acid, thioC₁₋₆alkylcarbonyl, thioC₁₋₆alkylaminocarbonyl, C₁₋₆alkyldisulfide, phenyldisulfide, —C(O)NH(CH₂)₁, —SO₃H, —C(O)NH(CH₂)₁₋₆—P(O)(OH)₂, —OR²³, —NH—R²³—C(O)NH—(CH₂)_{d}—R²³—S—(CH₂)_{d}—R²³, —(CH₂)_{d}—R²³, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted, C₁₋₆alkylthiourea substituted phenylurea or substituted phenylthiourea wherein the substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile,
where
d is 0-8;
R²³ is thioC₁₋₆alkylcarbonyl,
C₁₋₆alkyl,
substituted C₁₋₆alkyl
where the alkyl substituents are selected from one or more members of the group consisting of C₁₋₆alkyl, halo C₁₋₆alkyl, halogen, hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR²⁴ R²⁵)_{q}—(CHR²⁶)ₘ—SO₃H
where R²⁴, R²⁵, and R²⁶ are independently selected from the group consisting of hydrogen, halogen, hydroxyl, and C₁₋₆alkyl,
q is 1-6, and
m is 0-6
—(CH₂)ₙ—S—S—(CH₂)ₓNH—C(O)CR²⁷CH₂,
where R²⁷ is hydrogen or C₁₋₆alkyl,
n is 1-6, and
x is 1-6;
—(CR²⁸R²⁹)ₜ—(CHR³⁰)ᵤ—P(O)(OH)₂
where R²⁸, R²⁹, and R³⁰ are independently selected from the group consisting of hydrogen, halogen, hydroxyl, and C₁₋₆alkyl,
t is 1-6, and
u is 0-6;
phenyl;
benzyl;
pyridinyl;
pyrimidinyl;
pyrazinyl;
benzimidazolyl;
benzothiazolyl;
benzotriazolyl;
naphthaloyl;
quinolinyl;
indolyl;
thiadiazolyl;
triazolyl;
4-methylpiperidin-1-yl;
4-methylpiperazin-1-yl;
substituted phenyl;
substituted benzyl;
substituted pyridinyl;
substituted pyrimidinyl;
substituted pyrazinyl;
substituted benzimidazolyl;
substituted benzothiazolyl;
substituted benzotriazolyl;
substituted naphthaloyl;
substituted quinolinyl;
substituted indolyl;
substituted thiadiazolyl;
substituted triazolyl;
substituted 4-methylpiperidin-1-yl; or
substituted 4-methylpiperazin-1-yl,
wherein the substituents are selected from one or more members of the group consisting of C₁₋₆alkyl, haloC₁₋₆alkyl, halogen, sulfonic acid, phosphonic acid, hydroxyl, carboxylic acid, amine, amidine, N-(2-aminopyrimidine)sulfonyl, N-(aminopyridine)sulfonyl, N-(aminopyrazine)sulfonyl, N-(2-aminopyrimidine)carbonyl, N-(aminopyridine)carbonyl, N-(aminopyrazine)carbonyl, N-(2-aminopyrimidine)phosphonyl, N-(2-aminopyridine)phosphonyl, N-(aminopyrazine)phosphonyl, N-(aminobenzimidazolyl)sulfonyl, N-(aminobenzothiazolyl)sulfonyl, N-(aminobenzotriazolyl)sulfonyl, N-(aminoindolyl)sulfonyl, N-(aminothiazolyl)sulfonyl, N-(aminotriazolyl)sulfonyl, N-(amino-4-methylpiperidinyl)sulfonyl, N-(amino-4-methylpiperazinyl)sulfonyl, N-(aminobenzimidazolyl)carbonyl, N-(aminobenzothiazolyl)carbonyl, N-(aminobenzotriazolyl)carbonyl, N-(aminoindolyl)carbonyl, N-(aminothiazolyl)carbonyl, N-(aminotriazolyl)carbonyl, N-(amino-4-methylpiperidinyl)carbonyl, N-(amino-4-methylpiperazinyl)carbonyl, N-(2-aminobenzimidazolyl)phosphonyl, N-(2-aminobenzothiazolyl)phosphonyl, N-(2-aminobenzotriazolyl)phosphonyl, N-(2-aminoindolyl)phosphonyl, N-(2-aminothiazolyl)phosphonyl, N-(2-aminotriazolyl)phosphonyl, N-(amino-4-methylpiperidinyl) phosphonyl, N-(amino-4-methylpiperazinyl) phosphonyl, acetamide, nitrile, thiol, C₁₋₆alkyldisulfide, C₁₋₆alkylsulfide, phenyl disulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted C₁₋₆alkylthiourea, substituted phenylurea, and substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
w is 0-1;
Y is oxygen or sulfur;
R³¹ is hydrogen or C₁₋₆alkyl;
R³² is hydroxyl, sulfonic acid, phosphonic acid, carboxylic acid, thioC₁₋₆alkylcarbonyl, thioC₁₋₆alkylaminocarbonyl, —C(O)NH—(CH₂)_{d}—R³³, —O—R³³, —NH—R³³—S—(CH₂)_{d}—R³³, —(CH₂)_{d}—R³³, C₁₋₆alkyldisulfide, phenyldisulfide, urea, C₁₋₆alkylurea, phenylurea, thiourea, C₁₋₆alkylthiourea, phenylthiourea, C₁₋₆alkylamine, phenylamine, substituted C₁₋₆alkyldisulfide, substituted phenyldisulfide, substituted phenylurea, substituted C₁₋₆alkylamine, substituted phenylamine, substituted phenylthiourea, substituted C₁₋₆alkylurea or substituted C₁₋₆alkylthiourea wherein the substitutents are selected from the group consisting of C₁₋₆alkyl, haloC₁₋₆alkyl, halogen, hydroxyl, carboxylic acid, sulfonic acid, phosphonic acid, amine, amidine, acetamide, and nitrile
where
d is 0-8;
R³³ is thioC₁₋₆alkylcarbonyl,
C₁₋₆alkyl,
substituted C₁₋₆alkyl
where the alkyl substituents are selected from one or more members of the group consisting of C₁₋₆alkyl, halo C₁₋₆alkyl, halogen, hydroxyl, carboxylic acid, sulfonic acid, phosphonic acid, amine, amidine, acetamide, nitrile, thiol, C₁₋₆alkyldisulfide, C₁₋₆alkylsulfide, phenyldisulfide, urea, C₁₋₆alkylurea, phenylurea, thiourea, C₁₋₆alkylthiourea, phenylthiourea, substituted C₁₋₆alkyldisulfide, substituted phenyldisulfide, substituted C₁₋₆alkylurea, substituted phenylurea, substituted C₁₋₆alkylthiourea or substituted phenylthiourea
wherein the C₁₋₆alkyldisulfide, phenyldisulfide, C₁₋₆alkylurea, C₁₋₆alkylthiourea, phenylurea, and phenylthiourea substituents are selected from the group consisting of C₁₋₆alkyl, haloC₁₋₆alkyl, halogen, hydroxyl, carboxylic acid, sulfonic acid, phosphonic acid, amine, amidine, acetamide, and nitrile;
—(CR³⁴R³⁵)_{q}—(CHR³⁶)ₘ—SO₃H
where R³⁴, R³⁵, and R³⁶ are independently selected from the group consisting of hydrogen, halogen, hydroxyl, and C₁₋₆alkyl,
q is 1-6, and
m is 0-6;
—(CH₂)ₙ—S—S—(CH₂)ₓNH—C(O)CR³⁷CH₂,
where R³⁷ is hydrogen or C₁₋₆alkyl,
n is 1-6, and
x is 1-6;
—(CR³⁸R³⁹)ₜ—(CHR⁴⁰)ᵤ—P(O)(OH)₂
where R³⁸, R³⁹, and R⁴⁰ are independently selected from the group consisting of hydrogen, halogen, hydroxyl, and C₁₋₆alkyl,
t is 1-6, and
u is 0-6;
phenyl;
benzyl;
pyridinyl;
pyrimidinyl;
